(19) United States
(12) Patent Application Publication
(10) Pub. No.: US 2010/0247564 A1
(43) Pub. Date: Sep. 30, 2010

(54) METHODS AND COMPOSITIONS FOR THE USE OF SARGASSUM FUSIFORME FOR THE INHIBITION OF HIV-1 INFECTION

(76) Inventors: David Yue-wei Lee, Cambridge, MA (US); Mario Canki, Gulderland, NY (US)

Correspondence Address: CLARK & ELBING LLP, 101 FEDERAL STREET, BOSTON, MA, 02110, US

(21) Appl. No.: 12/601,072

(22) PCT Filed: May 23, 2008
(86) PCT No.: PCT/US08/06601

§ 371 Date: Jun. 11, 2010

Related U.S. Application Data
(60) Provisional application No. 60/931,619, filed on May 24, 2007.

(51) Int. Cl. A61K 36/03; A61P 31/18; A61P 31/22; A61K 31/20; A61K 31/201; C07C 57/03; C07C 53/00

(52) U.S. Cl. 424/195.17; 514/558; 514/560; 554/224; 554/223; 554/1

(57) ABSTRACT

The methods, kits, articles, and compositions of the invention feature a natural product (e.g., Sargassum fusiforme), an extract thereof, and fatty acid components of the extract (e.g., palmitic acid) for the treatment of a viral infection, e.g., HIV or herpes. The natural products used in the methods and compositions of the invention include brown algae, specifically algae of the Sargassum fusiforme species. The Sargassum fusiforme algae, extracts thereof, specific components of the extract, and related compounds of the invention may be used to treat or prevent HIV and herpes infections.

### BACKGROUND OF THE INVENTION

The high rate of human immunodeficiency virus (e.g., HIV-1 or HIV-2) mutation and increasing resistance to currently available antiretroviral (ARV) therapies highlight the need for new antiviral agents. Virus replication in the presence of ARV increases the likelihood and frequency of generating new multi-drug-resistant (MDR) HIV strains, as demonstrated by the observation that approximately 20% of all new HIV-1 infections are resistant to the currently available drugs. Consequently, concerted efforts toward the discovery and development of novel inhibitors of HIV infection and replication must persist if continued viral repression and virus eradication are to be achieved.

Much like HIV, herpes simplex virus (HSV) has demonstrated resistance against currently available therapies. HSV is transmitted upon contact with an infected person who is shedding virus from the skin, in saliva, or in secretions from the genitals. Treatment is available in the form of antiviral medications, such as nucleoside analogs, which reduce the duration of the symptoms of a HSV outbreak and accelerate healing. In the clinical setting, roughly 1-2% of the patients are infected with nucleoside-resistant HSV. However, in the immunocompromised patient population (e.g., transplant, AIDS, or cancer patients), the resistance rate can reach up to 10%.

Products derived from natural sources have been shown to inhibit the replication of certain viruses (e.g., HIV-1) during various stages of the virus life cycle and represent a potential source of novel therapeutic agents. For example, sulfated polysaccharides derived from sea algae have been tested for their ability to inhibit viral replication.

There exists a need in the art for new inhibitors of viral replication (e.g., replication of HIV-1, HIV-2, HSV-1, or HSV-2). The present invention addresses this issue and offers novel advantages over inhibitors known in the art.

### SUMMARY OF THE INVENTION

Applicants have discovered that brown algae (e.g., Sargassum fusiforme), an extract thereof, and fatty acid components of the extract (e.g., palmitic acid) can be useful for the treatment of, e.g., HIV infections. Applicants have also discovered that linoleic and oleic acid are potent reverse transcriptase inhibitors. The invention features palmitic acid derivatives, oleic acid derivatives, and linoleic acid derivatives, which can be used to treat viral infections, such as HIV and herpes infections.

In a first aspect, the invention features a method of treating an HIV or herpes infection in a subject in need thereof by administering to the subject a compound of formula (I), or a salt thereof, in an amount sufficient to treat the infection.

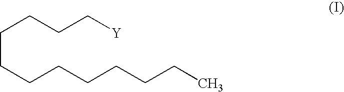

In formula (I) Y is selected from —CH₂CH₂CH₂COOH,

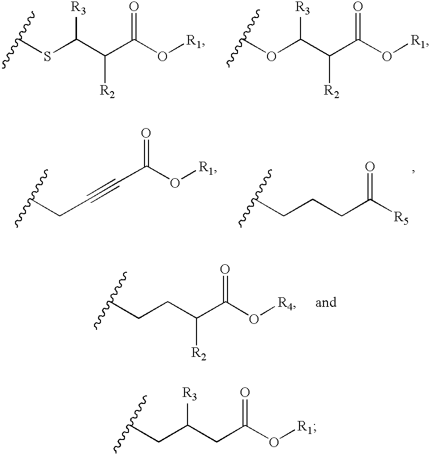

R₁ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; each of R₂ and R₃ is, independently, selected from H, —OR^{A}, —CH₃, —CH₂CH₃, halide, cyano, and nitro; R^{A} is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; R₄ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; and R₅ is selected from H, —CH₃, —CH₂CH₃, and CF₃. In certain embodiments, R₄ is selected from C₁₋₈ alkyl, C₂₋₈ alkenyl, C₂₋₈alkynyl, C₂₋₇ heterocyclyl, C₆₋₁₂ aryl, C₇₋₁₄ alkaryl, C₃₋₁₀ alkheterocyclyl. When R₄ is H, the compound of formula (I) can be selected from, without limitation, 2-fluoropalmitic acid, 2-bromopalmitic acid, 2-hydroxyhexadecanoic acid, 3-hydroxyhexadecanoic acid, and palmitic acid.

In certain embodiments, the method further includes administering to the subject a second compound selected from linoleic acid, salts thereof, and esters thereof, wherein the compound of formula (I) and the second compound are administered simultaneously or within 14 days of each other in amounts that together are sufficient to treat the infection. In other embodiments, the method further includes administering to the subject a second compound selected from oleic acid, salts thereof, and esters thereof, wherein the compound of formula (I) and the second compound are administered simultaneously or within 14 days of each other in amounts that together are sufficient to treat the infection.

In a related aspect, the invention features a method of inhibiting the transmission of HIV or herpes infection between a first subject and a second subject by topically applying to the first subject a compound of formula (I), or a salt thereof, in an amount sufficient to treat the infection.

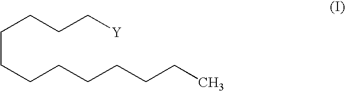

In formula (I) Y is selected from —CH₂CH₂CH₂COOH,

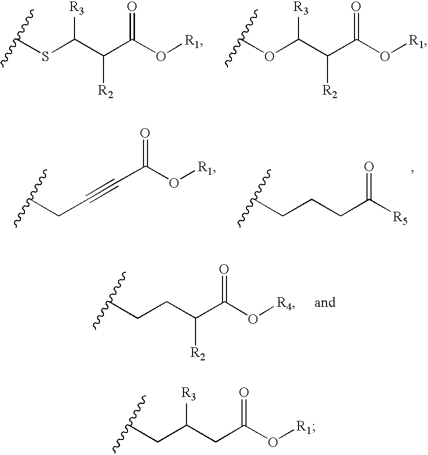

R₁ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; each of R₂ and R₃ is, independently, selected from H, —OR^{A}, —CH₃, —CH₂CH₃, halide, cyano, and nitro; R^{A} is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; R₄ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; and R₅ is selected from H, —CH₃, —CH₂CH₃, and CF₃. In certain embodiments, R₄ is selected from C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl. When R₄ is H, the compound of formula (I) can be selected from, without limitation, 2-fluoropalmitic acid, 2-bromopalmitic acid, 2-hydroxyhexadecanoic acid, 3-hydroxyhexadecanoic acid, and palmitic acid.

In certain embodiments, the method further includes administering to the subject a second compound selected from linoleic acid, salts thereof, and esters thereof, wherein the compound of formula (I) and the second compound are administered simultaneously or within 14 days of each other in amounts that together are effective to inhibit the transmission. In other embodiments, the method further includes administering to the subject a second compound selected from oleic acid, salts thereof, and esters thereof, wherein the compound of formula (I) and the second compound are administered simultaneously or within 14 days of each other in amounts that together are effective to inhibit the transmission.

The compound of formula (I) can be, for example, applied to the skin or a body cavity of the first subject. The compound of formula (I) can be formulated for topical administration as a foam, cream, wash, gel, spray, suppository, lotion, ointment, ovule, tampon, aerosol, or any other topical formulation described herein. In certain embodiments, the compound of formula (I) is applied as part of a contraceptive device (e.g., an intrauterine device, intravaginal barrier, intravaginal sponge, male condom, female condom, or any other contraceptive device described herein).

The invention also features an article including a compound of formula (I), or a salt thereof, in an amount sufficient to inhibit transmission of HIV or herpes to an individual wearing the article.

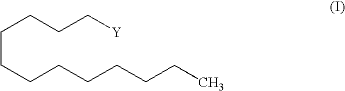

In formula (I) Y is selected from —CH₂CH₂CH₂COOH,

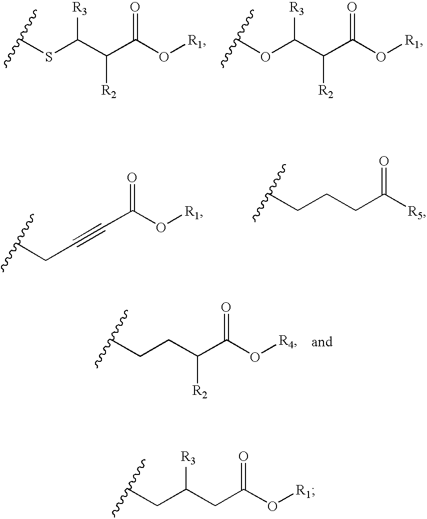

R₁ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; each of R₂ and R₃ is, independently, selected from H, —OR^{A}, —CH₃, —CH₂CH₃, halide, cyano, and nitro; R^{A} is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; R₄ is selected from H, C₁₋₈ alkyl, C₂₋₈ alkenyl, C₂₋₈alkynyl, C₂₋₇ heterocyclyl, C₆₋₁₂ aryl, C₇₋₁₄ alkaryl, C₃₋₁₀ alkheterocyclyl, and C₁₋₈ heteroalkyl; and R₅ is selected from H, —CH₃, —CH₂CH₃, and CF₃. In certain embodiments, R₄ is selected from C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl. When R₄ is H, the compound of formula (I) can be selected from, without limitation, 2-fluoropalmitic acid, 2-bromopalmitic acid, 2-hydroxyhexadecanoic acid, 3-hydroxyhexadecanoic acid, and palmitic acid.

The article can be, without limitation, selected from a glove, intrauterine device, vaginal dispenser, vaginal ring, intravaginal barrier-type device, intravaginal sponge, male condom, female condom, and any other article described herein. In certain embodiments, the article further includes a second compound selected from linoleic acid, salts thereof, and esters thereof, wherein the compound of formula (I) and the second compound are present in amounts that together are effective to inhibit the transmission. In other embodiments, the article further includes a second compound selected from oleic acid, salts thereof, and esters thereof, wherein the compound of formula (I) and the second compound are present in amounts that together are effective to inhibit the transmission.

The invention further features a pharmaceutical composition formulated for topical administration including from about 1% to about 50% (w/w) of a compound of formula (I), or a salt thereof.

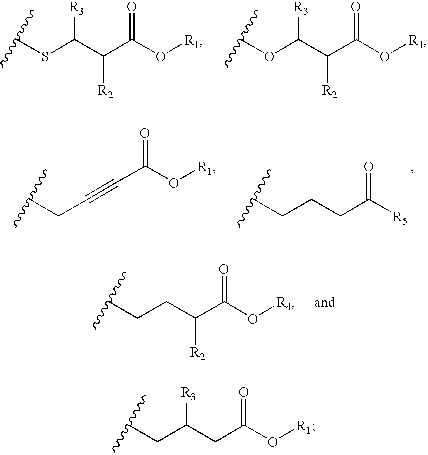

In formula (I) Y is selected from —CH₂CH₂CH₂COOH,

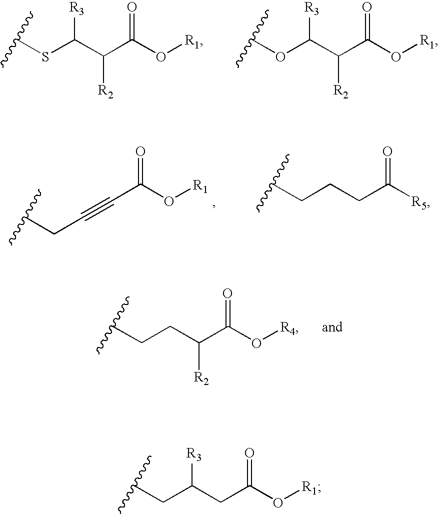

R₁ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; each of R₂ and R₃ is, independently, selected from H, —OR^{A}, —CH₃, —CH₂CH₃, halide, cyano, and nitro; R^{A} is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; R₄ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; and R₅ is selected from H, —CH₃, —CH₂CH₃, and CF₃. In certain embodiments, R₄ is selected from C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl. When R₄ is H, the compound of formula (I) can be selected from, without limitation, 2-fluoropalmitic acid, 2-bromopalmitic acid, 2-hydroxyhexadecanoic acid, 3-hydroxyhexadecanoic acid, and palmitic acid. In certain embodiments, the pharmaceutical composition includes from about 1% to 65%, 1% to 45%, 1% to 35%, 1% to 25%, 1% to 15%, 1% to 10%, 2% to 5%, 2% to 15%, 2% to 25%, 2% to 35%, 2% to 45%, 5% to 15%, 5% to 25%, 5% to 35%, 5% to 45%, 10% to 15%, 10% to 25%, 10% to 35%, or 15% to 35% (w/w) of a compound of formula (I), or a salt thereof.

In certain embodiments, the pharmaceutical composition further includes from about 1% to about 20%, 1% to 65%, 1% to 45%, 1% to 35%, 1% to 25%, 1% to 15%, 1% to 10%, 2% to 5%, 2% to 15%, 2% to 25%, 2% to 35%, 2% to 45%, 5% to 15%, 5% to 25%, 5% to 35%, 5% to 45%, 10% to 15%, 10% to 25%, 10% to 35%, or 15% to 35% (w/w) of a second compound selected from linoleic acid, salts thereof, and esters thereof. In other embodiments, the pharmaceutical composition includes from about 1% to about 20%, 1% to 65%, 1% to 45%, 1% to 35%, 1% to 25%, 1% to 15%, 1% to 10%, 2% to 5%, 2% to 15%, 2% to 25%, 2% to 35%, 2% to 45%, 5% to 15%, 5% to 25%, 5% to 35%, 5% to 45%, 10% to 15%, 10% to 25%, 10% to 35%, or 15% to 35% (w/w) of a second compound selected from oleic acid, salts thereof, and esters thereof.

The pharmaceutical composition formulated for topical administration can be formulated, without limitation, as a powder, a solution, a gel, a paste, an ointment, a cream, a foam, a lotion, a plaster, a suppository, an enema, a spray, an aerosol, or any other topical form described herein.

The invention further features a kit including (a)a pharmaceutical composition including a compound of formula (I), or a salt thereof, in an amount sufficient to treat HIV or herpes when administered to a subject; and (b) instructions for administering the composition to a subject infected with HIV or herpes.

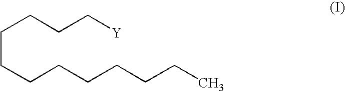

In formula (I) Y is selected from —CH₂CH₂CH₂COOH,

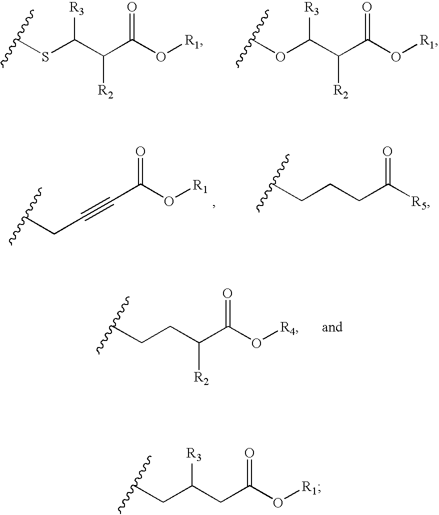

R₁ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; each of R₂ and R₃ is, independently, selected from H, —OR^{A}, —CH₃, —CH₂CH₃, halide, cyano, and nitro; R^{A} is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; R₄ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; and R₅ is selected from H, —CH₃, —CH₂CH₃, and CF₃. In certain embodiments, R₄ is selected from C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl. When R₄ is H, the compound of formula (I) can be selected from, without limitation, 2-fluoropalmitic acid, 2-bromopalmitic acid, 2-hydroxyhexadecanoic acid, 3-hydroxyhexadecanoic acid, and palmitic acid.

The invention further features a kit including (a) a pharmaceutical composition including a compound of formula (I), or a salt thereof, in an amount effective to inhibit the transmission of HIV or herpes when administered to a subject; and (b) instructions for administering the composition to a subject at risk of being infected with HIV or herpes.

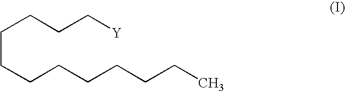

In formula (I) Y is selected from —CH₂CH₂CH₂COOH,

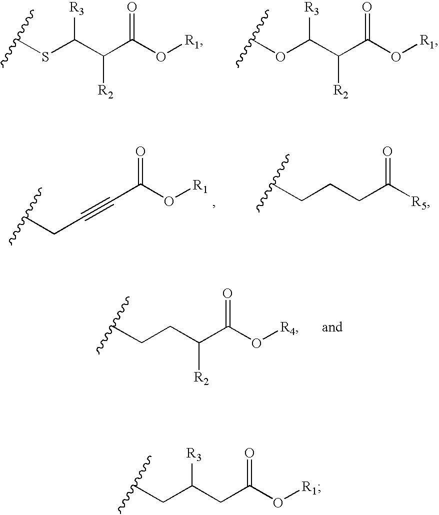

R₁ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; each of R₂ and R₃ is, independently, selected from H, —OR^{A}, —CH₃, —CH₂CH₃, halide, cyano, and nitro; R^{A} is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; R₄ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; and R₅ is selected from H, —CH₃, —CH₂CH₃, and CF₃. In certain embodiments, R₄ is selected from C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl. When R₄ is H, the compound of formula (I) can be selected from, without limitation, 2-fluoropalmitic acid, 2-bromopalmitic acid, 2-hydroxyhexadecanoic acid, 3-hydroxyhexadecanoic acid, and palmitic acid.

The any of the above kits of the invention, the pharmaceutical composition can further include a second compound selected from linoleic acid, salts thereof, and esters thereof and oleic acid, salts thereof, and esters thereof.

The invention further features a method of treating an HIV or herpes infection in a subject in need thereof by administering to the subject linoleic acid, or a salt or ester thereof, in an amount sufficient to treat the infection.

The invention also features a method of treating an HIV or herpes infection in a subject in need thereof by administering to the subject oleic acid, or a salt or ester thereof, in an amount sufficient to treat the infection.

The invention also features a method of treating an HIV or herpes infection in a subject in need thereof by administering to the subject a mixture of (i) oleic acid, or a salt or ester thereof, and (ii) linoleic acid, or a salt or ester thereof, simultaneously or within 14 days of each other in amounts that together are sufficient to treat the infection.

In a related aspect, the invention features a method of inhibiting the transmission of HIV or herpes infection between a first subject and a second subject by topically applying to the first subject linoleic acid, or a salt or ester thereof, in an amount effective to inhibit the transmission.

The invention also features a method of inhibiting the transmission of HIV or herpes infection between a first subject and a second subject by topically applying to the first subject oleic acid, or a salt or ester thereof, in an amount effective to inhibit the transmission.

The invention further features a method of inhibiting the transmission of HIV or herpes infection between a first subject and a second subject by topically applying to the first subject a mixture of (i) oleic acid, or a salt or ester thereof, and (ii) linoleic acid, or a salt or ester thereof, simultaneously or within 14 days of each other in amounts that together are effective to inhibit the transmission.

The invention features a kit including (a) a pharmaceutical composition including linoleic acid, or a salt or ester thereof, in an amount sufficient to treat HIV or herpes when administered to a subject; and (b) instructions for administering the composition to a subject infected with HIV or herpes.

The invention also features a kit including (a) a pharmaceutical composition including oleic acid, or a salt or ester thereof, in an amount sufficient to treat HIV or herpes when administered to a subject; and (b) instructions for administering the composition to a subject infected with HIV or herpes.

In a related aspect, the invention features a kit including (a) a pharmaceutical composition including linoleic acid, or a salt or ester thereof, in an amount effective to inhibit the transmission of HIV or herpes when administered to a subject; and (b) instructions for administering the composition to a subject at risk of being infected with HIV or herpes.

The invention further features a kit including (a) a pharmaceutical composition including oleic acid, or a salt or ester thereof, in an amount effective to inhibit the transmission of HIV or herpes when administered to a subject; and (b) instructions for administering the composition to a subject at risk of being infected with HIV or herpes.

In any of the above methods, kits, articles, and compositions, the compound of formula (I) can be selected from palmityl trifluoromethyl ketone, 2-heptadecanone, 3-octadecanone, 2-hexadecynoic acid or an ester thereof, 3-dodecyloxypropionic acid or an ester thereof, 3-dodecylthiopropionic acid or an ester thereof, palmitic acid or an ester thereof, 3-hydroxyhexadecanoic acid or an ester thereof, esters of 2-hydroxyhexadecanoic acid, esters of 2-fluoropalmitic acid, esters of 2-bromopalmitic acid, and any compound of any of formulas (I)-(VII) described herein.

In any of the above methods, kits, articles, and compositions the ester (e.g., an ester of any of formulas (I)-(VII), a palmitic acid ester, a fatty acid ester, a linoleic acid ester, or an oleic acid ester) is selected from methyl ester, ethyl ester, propyl ester, or any other ester described herein.

The invention features a method of treating HIV-1 infection in a subject in need thereof by administering brown algae to the subject in an amount sufficient to treat the infection.

In a related aspect, the invention features a method of treating HIV-1 infection in a subject in need thereof by administering an extract of brown algae to the subject in an amount sufficient to treat the infection.

In an embodiment of any of the above aspects, the brown alga is selected from Sargassum spp. In certain embodiments the Sargassum spp. is Sargassum fusiforme.

The invention further features a method of treating HIV-1 infection in a subject in need thereof by administering an isolated bioactive fraction of a Sargassum fusiforme extract to the subject in an amount sufficient to treat the infection. In certain embodiments the isolated bioactive fraction includes fatty acids. Desirably, the isolated bioactive fraction includes palmitic acid, oleic acid, and linoleic acid. The extract can be, for example, an aqueous extract or an aqueous acetone extract.

The invention also features a method of treating HIV-1 infection in a subject in need thereof by administering substantially pure fatty acid, or a salt or ester thereof, to the subject in an amount sufficient to treat the infection. In certain embodiments the fatty acid is palmitic acid, oleic acid, linoleic acid, or a salt or ester thereof. In still other embodiments the fatty acid, or salt or ester thereof, is isolated from an extract of Sargassum fusiforme.

The invention features a pharmaceutical composition including an isolated bioactive fraction of a Sargassum fusiforme extract and a pharmaceutically acceptable excipient. In certain embodiments the isolated bioactive fraction comprises palmitic acid, oleic acid, linoleic acid, or a salt or ester thereof.

The invention also features a pharmaceutical composition including substantially pure palmitic acid, or a salt or ester thereof, and a pharmaceutically acceptable excipient. In certain embodiments the palmitic acid, oleic acid, linoleic acid, or salt or ester thereof, is isolated from an extract of Sargassum fusiforme.

The invention further features a kit including (a) a pharmaceutical composition including Sargassum fusiforme; and (b) instructions for administering the composition to a subject infected with HIV-1.

In a related aspect, the invention features a kit including (a) a pharmaceutical composition including an isolated bioactive fraction of a Sargassum fusiforme extract; and (b) instructions for administering the composition to a subject infected with HIV-1. In certain embodiments the isolated bioactive fraction includes palmitic acid, oleic acid, linoleic acid, or a salt or ester thereof.

The invention further features a kit including (a) a pharmaceutical composition including substantially pure palmitic acid, oleic acid, linoleic acid, or a salt or ester thereof; and (b) instructions for administering the composition to a patient infected with HIV-1. In certain embodiments the palmitic acid, oleic acid, linoleic acid, or salt or ester thereof, is isolated from an extract of Sargassum fusiforme.

The invention further features a dietary supplement or nutraceutical including: (a) a vitamin selected from vitamin C, vitamin D, vitamin E, vitamin K, folate, vitamin B6, and vitamin B 12; and (b) a compound of formula (I), oleic acid or a salt or ester thereof, linoleic acid or a salt or ester thereof, or an extract of the invention.

The invention also features a dietary supplement or nutraceutical including: (a) a mineral selected from calcium, chromium, copper, fluoride, iodine, iron, magnesium, manganese, molybdenum, phosphorus, potassium, selenium, sodium, and zinc; and (b) a compound of formula (I), oleic acid or a salt or ester thereof, linoleic acid or a salt or ester thereof, or an extract of the invention.

The invention also features a dietary supplement or nutraceutical including: (a) an amino acid selected from isoleucine, leucine, lysine, methionine, phenylalanine, threonine, tryptophan, and valine; and (b) a compound of formula (I), oleic acid or a salt or ester thereof, linoleic acid or a salt or ester thereof, or an extract of the invention.

In still another related aspect, the invention features a dietary supplement or nutraceutical including: (a) an herb selected from angelica, astragalus, avena sativa, bayberry bark, billberry, black cohosh, black haw, black walnut, blessed thistle, blue cohosh, blue vervain, buchu, buckthorn, burdock, cascara sagada, casteberry, cayenne, chamomille, chaparral, chaste tree, chickweed, cloves, coltsfoot, comphrey root, cornsilk, cough calm, crampbark, damiana, dandelion, dandelion root, dill seed, dong quai, echinacea, elecampane, essiac, eucalyptus, fennel, fenugreek, gentian, ginger, ginkgo, ginseng, goldenseal, gota kola, guarana, hawthorne berry, hops, horehound, horsetail, hydrangea, hyssop, kelp, kola nut, licorice, lobelia, maca, marshmallow, motherwort, muira puama, mullien, myrrh, nettle, oatstraw, oregon grape root, parsley, passion flower, pau d' arco, pepermint, plantain, pleurisy root, prickley ash bark, red clover, red raspberry, sarsaparilla, saw palmetto, schizandra, scullcap, sheep sorrel, slippery elm, squawvine, St. Johns wort, tumeric, turkey rhubarb, valerian, white willow bark, wild cherry bark, wild yam, yarrow, yellow dock, yohimbi, and extracts thereof; and (b) a compound of formula (I), oleic acid or a salt or ester thereof, linoleic acid or a salt or ester thereof, or an extract of the invention.

The invention further features a dietary supplement or nutraceutical formulated in unit dosage form containing from 10 mg to 2 g a compound of formula (I), oleic acid or a salt or ester thereof, linoleic acid or a salt or ester thereof, or an extract of the invention. In certain embodiments, the dietary supplement or nutraceutical in a unit dosage form contains from about 10 mg to 1 g, 10 mg to 500 mg, 10 mg to 250 mg, 100 mg to 500 mg, 50 mg to 2 g, 50 mg to 1 g, 50 mg to 500 mg, or100 mg to 500 mg of the compound of formula (I), oleic acid or a salt or ester thereof, or linoleic acid or a salt or ester thereof.

In certain embodiments of the dietary supplements of the invention, the dietary supplement or nutraceutical is formulated in unit dosage form as a tablet, pill, capsule, or caplet. In still other embodiments, the dietary supplement or nutraceutical is formulated as a liquid or a powder containing from 5% to 75% (w/w) of the compound of formula (I), oleic acid or a salt or ester thereof, or linoleic acid or a salt or ester thereof. Desirably, the dietary supplement or nutraceutical contains between 5% and 50%, 5% and 40%, 10% and 65%, 20% and 65%, 20% and 50%, or 30% and 75% (w/w) compound of formula (I), oleic acid or a salt or ester thereof, or linoleic acid or a salt or ester thereof.

The invention also features a kit, including: (i) a dietary supplement or nutraceutical of the invention; and (ii) instructions for administering the dietary supplement or nutraceutical to a subject.

Any of the above methods can further include administering to the subject an additional antiviral agent simultaneously or within 14 days.

Any of the above kits, articles, and compositions can further include an additional antiviral agent.

Additional antiviral agents are non-fatty acid therapeutics. Such an antiviral agent can be, for example, a protease inhibitor, a reverse transcriptase inhibitor, an integrase inhibitor, a CCR5 antagonist, a fusion inhibitor, or a second maturation inhibitor. The additional antiviral agent can be, without limitation, azidovudine (AZT), didanosine (dideoxyinosine, ddI), d4T, zalcitabine (dideoxycytosine, ddC), nevirapine, lamivudine (epivir, 3TC), saquinavir (Invirase), ritonavir (Norvir), indinavir (Crixivan), delavirdine (Rescriptor), or any antiviral agent described herein.

In any of the kits of the invention, the additional antiviral agent can be included in the pharmaceutical composition within the kit, be included in the kit as a separately formulated composition, or the kit can simply include instructions for administration in a combination therapy that includes a second antiviral agent.

The methods, kits, articles, and compositions of the invention may be used to treat, or prevent transmission of, retroviral infections, including, e.g., HIV-1, HIV-2, human T-cell leukemia virus type 1, human T-cell leukemia virus type 2, feline immunodeficiency virus, or feline leukemia virus.

The methods, kits, articles, and compositions of the invention may also be used to treat, or prevent transmission of, herpes virus, including, e.g., herpes simplex virus type 1 (HSV-1), herpes simplex virus type 2 (HSV-2), varicella-zoster virus (VZV), cytomegalovirus (CMV), human herpes virus 6 (HHV-6), human herpes virus 7 (HHV-7), Epstein-Barr virus (EBV) and Kaposi's herpes virus (HHV-8).

In certain embodiments of any of the methods, kits, articles, and compositions of the invention including an ester (e.g., of any of formulas (I)-(VII), palmitic acid, linoleic acid, or oleic acid), the ester is not a fatty acid ester of a therapeutically active substance.

In certain embodiments of any of the invention, the compounds of the invention (e.g., of any of formulas (I)-(VII), palmitic acid, linoleic acid, or oleic acid) modulate the activity of (e.g., inhibit) reverse transcriptase (RT).

In another embodiment of any of the methods, kits, articles, and compositions of the invention including an optically active compound of any of formulas (I)-(VII) (e.g., 2-fluoropalmitic acid, 2-bromopalmitic acid, 2-hydroxyhexadecanoic acid, 3-hydroxyhexadecanoic acid, and salts and esters thereof), the compound may be part of an enantiomeric mixture or enriched in the R isomer or the S isomer such that the enantiomeric excess is at least 10%, 20%, 30%, 40%, 50%, 60%, 70%, 80%, 90%, 95%, or 98%. Enantiomeric excess can be prepared either by selecting an enantiospecific synthetic route for the synthesis of such compounds, or using enantioselective purification techniques known in the art.

As used herein, an “amount sufficient” refers to the amount of a therapeutic (e.g., Sargassum fusiforme, an extract thereof, compounds of the invention, and combinations of the invention) sufficient to achieve a desired result. The amount sufficient will vary depending upon a variety of parameters, e.g., the condition being treated, the site being treated, the therapeutic being administered, and the delivery vehicle employed. An effective amount can be determined for any given set of conditions using standard methods. Based upon these results, a vehicle may be prepared which releases the therapeutic at a rate that produces the desired effect.

As used herein, the term “antiviral agent” refers to a substance capable of destroying or suppressing the replication of viruses (e.g., HIV-1 or herpes virus). The antiviral agent may be, e.g., azidovudine (AZT), didanosine (dideoxyinosine, ddI), d4T, zalcitabine (dideoxycytosine, ddC), nevirapine, lamivudine (epivir, 3TC), saquinavir (Invirase), ritonavir (Norvir), indinavir (Crixivan), or delavirdine (Rescriptor), among others.

As used herein, the term “brown algae” refers to any algae of the class Phaeophyceae, particularly algae of the genus Sargassum. Examples of brown algae include, e.g., Sargassum fusiforme, Sargassum aquifolium, Sargassum crassifolium, Sargassum duplicatum, Sargassum filicinum, Sargassum filipendula, Sargassum gramminifolium, Sargassum henslowianum, Sargassum horneri, Sargassum ilicifolium, Sargassum mcclurei, Sargassum muticum, Sargassum myriocystum, Sargassum natans, Sargassum oligocystum, Sargassum patens, Sargassum polycystum, Sargassum serratifolium, Sargassum siliquosum, Sargassum wightii, Sargassum vachelliannum, Colpomenia sinuosa, Ecklonia cava, Forsythia suspense, Laminaria digitata, Laminaria japonica, Macrocystis pyrifera, Padina arborescens, Petalonia fascia, Pilayella littoralis, Prunella vulgaris, Scytosiphon lomentaria, and Undaria pinnatifida.

As used herein, the term “extract” refers to a preparation that contains a single chemical component or multiple chemical components of, e.g., brown algae. The component may be, e.g., a fatty acid (e.g., palmitic acid, linoleic acid, and/or oleic acid). The extract may be prepared by chemical, physical, or mechanical separation methods. For example, the brown algae may be ground into particulates and soaked in an aqueous or organic solution. Components that are soluble in the aqueous or organic solution will be extracted from the brown algae. The extract may be concentrated or subjected to further fractionation.

As used herein, the term “isolated bioactive fraction” refers to a portion of an aqueous or aqueous acetone (e.g., 70% acetone) extract of brown algae (e.g., Sargassum fusiforme) having antiviral activity. Fractions of the extract are isolated using any separation method known in the art, e.g., column chromatography. The term “bioactive” refers to the ability of the fraction to inhibit any step of viral infection, e.g., virus replication. Bioactivity may be determined using any method known in the art, e.g., plaque assays or reporter gene technologies (e.g., monitoring the expression of luciferase or green fluorescent protein).

By “pharmaceutical composition” is meant any composition that contains at least one biologically active agent and is suitable for administration to a patient. For the purposes of this invention, pharmaceutical compositions suitable for delivering a therapeutic can include, e.g., tablets, gelcaps, capsules, pills, solutions, delivery devices, or implants. Any of these formulations can be prepared by well-known and accepted methods of art. See, for example, Remington: The Science and Practice of Pharmacy (21^{st} ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

A “pharmaceutically acceptable excipient,” as used herein, refers to an excipient that is physiologically acceptable to the treated patient while retaining the therapeutic properties of the agent or compound (e.g., Sargassum fusiforme or an extract thereof (e.g., palmitic acid, linoleic acid, oleic acid, or any combination thereof)) with which it is administered. One exemplary pharmaceutically acceptable excipient is physiological saline. Other physiologically acceptable excipients and their formulations are known to one skilled in the art.

By “fatty acid” is meant any carboxylic acid with an unbranched, aliphatic carbon backbone. The fatty acid may be saturated (e.g., does not contain any double bonds), unsaturated (e.g., contains double bonds), monounsaturated (e.g., contains a single double bond), and polyunsaturated (e.g., contains multiple double bonds) fatty acids. Exemplary fatty acids include, e.g., decanoic acid (DA), undecanoic acid (UA), dodecanoic acid (lauric acid), myristic acid (MA), palmitic acid (PA), stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, butyric acid, caproic acid, caprylic acid, capric acid, behenic acid, docosahexaenoic acid, erucic acid, eicosapentaenoic acid, and cerebronic acid.

As used herein, the term “salt” refers to any pharmaceutically acceptable salt of a saturated fatty acid. Salts include, without limitation, non-toxic acid addition salts to organic bases (e.g., meglumine salts) or metal salts and/or complexes that are commonly used in the pharmaceutical industry. Examples of metal salts include, without limitation, sodium, magnesium, and potassium salts. Examples of metal complexes include, without limitation, calcium, zinc, and iron complexes of fatty acids.

As used herein, the term “ester” refers to a fatty acid derivative formed by replacing the acidic proton of a saturated fatty acid with an organic group. Esters include, without limitation, derivatives in which the organic group is selected from C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl.

In the generic descriptions of compounds of this invention, the number of atoms of a particular type in a substituent group is generally given as a range, e.g., an alkyl group containing from 1 to 7 carbon atoms or C₁₋₇ alkyl. Reference to such a range is intended to include specific references to groups having each of the integer number of atoms within the specified range. For example, an alkyl group from 1 to 7 carbon atoms includes each of C₁, C₂, C₃, C₄, C₅, C₆, and C₇.

By “C₁₋₈ alkyl” is meant straight chain, branched chain, and of cyclic groups, i.e., cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups. The C₁₋₈ alkyl group may be substituted or unsubstituted. Exemplary substituents include alkoxy, aryloxy, sulfhydryl, alkylthio, arylthio, halide, hydroxyl, fluoroalkyl, perfluoralkyl, cyano, nitrilo, NH-acyl, amino, aminoalkyl, disubstituted amino, quaternary amino, hydroxyalkyl, carboxyalkyl, and carboxyl groups. C₁₋₈ alkyls include, without limitation, methyl; ethyl; n-propyl; isopropyl; cyclopropyl; cyclopropylmethyl; cyclopropylethyl; n-butyl; iso-butyl; sec-butyl; tert-butyl; cyclobutyl; cyclobutylmethyl; cyclobutylethyl; n-pentyl; cyclopentyl; cyclopentylmethyl; cyclopentylethyl; 1-methylbutyl; 2-methylbutyl; 3-methylbutyl; 2,2-dimethylpropyl; 1-ethylpropyl; 1,1-dimethylpropyl; 1,2-dimethylpropyl; 1-methylpentyl; 2-methylpentyl; 3-methylpentyl; 4-methylpentyl; 1,1-dimethylbutyl; 1,2-dimethylbutyl; 1,3-dimethylbutyl; 2,2-dimethylbutyl; 2,3-dimethylbutyl; 3,3-dimethylbutyl; 1-ethylbutyl; 2-ethylbutyl; 1,1,2-trimethylpropyl; 1,2,2-trimethylpropyl; 1-ethyl-1-methylpropyl; 1-ethyl-2-methylpropyl; and cyclohexyl.

By “C₂₋₈ alkenyl” is meant a branched or unbranched hydrocarbon group containing one or more double bonds and having from 2 to 8 carbon atoms. A C₂₋₈ alkenyl may optionally include monocyclic or polycyclic rings, in which each ring desirably has from three to six members. The C₂₋₈ alkenyl group may be substituted or unsubstituted. Exemplary substituents include alkoxy, aryloxy, sulfhydryl, alkylthio, arylthio, halide, hydroxyl, fluoroalkyl, perfluoralkyl, cyano, nitrilo, NH-acyl, amino, aminoalkyl, disubstituted amino, quaternary amino, hydroxyalkyl, carboxyalkyl, and carboxyl groups. C₂₋₈ alkenyls include, without limitation, vinyl; allyl; 2-cyclopropyl-1-ethenyl; 1-propenyl; 1-butenyl; 2-butenyl; 3-butenyl; 2-methyl-1-propenyl; 2-methyl-2-propenyl; 1-pentenyl; 2-pentenyl; 3-pentenyl; 4-pentenyl; 3-methyl-1-butenyl; 3-methyl-2-butenyl; 3-methyl-3-butenyl; 2-methyl-1-butenyl; 2-methyl-2-butenyl; 2-methyl-3-butenyl; 2-ethyl-2-propenyl; 1-methyl-1-butenyl; 1-methyl-2-butenyl; 1-methyl-3-butenyl; 2-methyl-2-pentenyl; 3-methyl-2-pentenyl; 4-methyl-2-pentenyl; 2-methyl-3-pentenyl; 3-methyl-3-pentenyl; 4-methyl-3-pentenyl; 2-methyl-4-pentenyl; 3-methyl-4-pentenyl; 1,2-dimethyl-1-propenyl; 1,2-dimethyl-1-butenyl; 1,3-dimethyl-1-butenyl; 1,2-dimethyl-2-butenyl; 1,1-dimethyl-2-butenyl; 2,3-dimethyl-2-butenyl; 2,3-dimethyl-3-butenyl; 1,3-dimethyl-3-butenyl; 1,1-dimethyl-3-butenyl and 2,2-dimethyl-3-butenyl.

By “C₂₋₈ alkynyl” is meant a branched or unbranched hydrocarbon group containing one or more triple bonds and having from 2 to 8 carbon atoms. A C₂₋₈ alkynyl may optionally include monocyclic, bicyclic, or tricyclic rings, in which each ring desirably has five or six members. The C₂₋₈ alkynyl group may be substituted or unsubstituted. Exemplary substituents include alkoxy, aryloxy, sulfhydryl, alkylthio, arylthio, halide, hydroxy, fluoroalkyl, perfluoralkyl, cyano, nitrilo, NH-acyl, amino, aminoalkyl, disubstituted amino, quaternary amino, hydroxyalkyl, carboxyalkyl, and carboxyl groups. C₂₋₈ alkynyls include, without limitation, ethynyl, 1-propynyl, 2-propynyl, 1-butynyl, 2-butynyl, 3-butynyl, 1-pentynyl, 2-pentynyl, 3-pentynyl, 4-pentynyl, 5-hexene-1-ynyl, 2-hexynyl, 3-hexynyl, 4-hexynyl, 5-hexynyl; 1-methyl-2-propynyl; 1-methyl-2-butynyl; 1-methyl-3-butynyl; 2-methyl-3-butynyl; 1,2-dimethyl-3-butynyl; 2,2-dimethyl-3-butynyl; 1-methyl-2-pentynyl, 2-methyl-3-pentynyl; 1-methyl-4-pentynyl; 2-methyl-4-pentynyl; and 3-methyl-4-pentynyl.

By “C₂₋₇ heterocyclyl” is meant a stable 5- to 7-membered monocyclic or 7- to 14-membered bicyclic heterocyclic ring which is saturated partially unsaturated or unsaturated (aromatic), and which consists of 2 to 7 carbon atoms and 1, 2, 3 or 4 heteroatoms independently selected from the group consisting of N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, cyano, nitrilo, NH-acyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary subsituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 7 to 14 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₈ heteroalkyl” is meant a branched or unbranched alkyl, alkenyl, or alkynyl group having from 1 to 8 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, cyano, nitrilo, NH-acyl, amino, aminoalkyl, disubstituted amino, quaternary amino, hydroxyalkyl, hydroxyalkyl, carboxyalkyl, and carboxyl groups. Examples of C₁₋₈ heteroalkyls include, without limitation, methoxymethyl and ethoxyethyl.

By “halide” is meant bromine, chlorine, iodine, or fluorine.

By “substantially pure” is meant a compound or molecule that has been separated from the components that naturally accompany it. Typically, the compound is substantially pure when it is at least 50, 55, 60, 65, 70, 75, 80, 85, 90, 95, or 99%, by weight, free from other naturally occurring components with which it is naturally associated. Purity can be measured by any appropriate method, e.g., by column chromatography, mass spectrometry, or HPLC analysis.

As used herein, the term “treating” refers to administering a composition of the invention, such as a compound of formula I, oleic acid and salts or esters thereof, linoleic acid and salts or esters thereof, a combination therapy of the invention, brown algae (e.g., Sargassum fusiforme), brown algae extract (e.g., Sargassum fusiforme extract), an isolated bioactive fraction of brown algae extract (e.g., isolated bioactive fraction of S. fusiforme extract), or a fatty acid (e.g., palmitic acid) for prophylactic and/or therapeutic purposes. To “prevent or inhibit disease” refers to prophylactic treatment of a subject who is not yet ill, but who is susceptible to, or otherwise at risk of, a particular disease. To “treat disease” or use for “therapeutic treatment” refers to administering treatment to a subject already suffering from a disease to improve or stabilize the subject's condition. Thus, in the claims and embodiments, treating is the administration to a subject either for therapeutic or prophylactic purposes.

As used herein, the terms “inhibit transmission” and “inhibiting transmission” refer to a 5%, 10%, 20%, 30%, 40%, or even 50% reduction in the transmission rate of HIV or herpes to an individual engaged in an activity placing them at risk of infection while undergoing a therapy of the invention (e.g., a subject taking Sargassum fusiforme, an extract thereof, a compound of the invention, or a combination of the invention) in comparison to the transmission rate observed for the same individual engaged in the same risky activity, but in the absence of the therapy.

As used herein, an “amount effective” refers to the amount of a therapeutic (e.g., Sargassum fusiforme, an extract thereof, compounds of the invention, and combinations of the invention) sufficient to achieve a desired result. The amount effective will vary depending upon a variety of parameters, e.g., the condition being treated, the site being treated, the therapeutic being administered, and the delivery vehicle employed. An effective amount can be determined for any given set of conditions using standard methods. Based upon these results, a vehicle may be prepared which releases the therapeutic at a rate that produces the desired effect.

By “oleic acid ester” is meant a compound of formula (A).

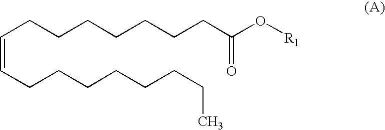

R₁ of formula (A) is selected from C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl. R₁ can be any organic group (e.g., methyl, ethyl, etc.) recited herein. In certain embodiments, R₁ is selected from C₁₋₄ alkyl, C₂₋₄ alkenyl, C₂₋₄ alkynyl, C₂₋₅ heterocyclyl, and C₁₋₄ heteroalkyl. R₁ can be any organic group described herein.

By “linoleic acid ester” is meant a compound of formula (B).

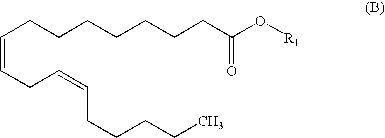

R₁ of formula (B) is selected from C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl. R₁ can be any organic group (e.g., methyl, ethyl, etc.) recited herein. In certain embodiments, R₁ is selected from C₁₋₄ alkyl, C₂₋₄ alkenyl, C₂₋₄ alkynyl, C₂₋₅ heterocyclyl, and C₁₋₄ heteroalkyl. R₁ can be any organic group described herein.

The term “herpes” refers to viruses of the Herpesviridae family. These viruses are large, have a double-strand DNA (dsDNA) genome of about 80-250 kilobases (kb) and are found in a wide range of host systems. About 100 herpesviruses have been isolated in various animal species, including the human species. Human herpes viruses that can be treated, and for which transmission can be inhibited, include, without limitation, herpes simplex virus type 1 (HSV-1), herpes simplex virus type 2 (HSV-2), varicella-zoster virus (VZV), cytomegalovirus (CMV), human herpes virus 6 (HHV-6), human herpes virus 7 (HHV-7), Epstein-Barr virus (EBV) and Kaposi's herpes virus (HHV-8).

The term “reverse transcriptase” (RT) refers to RNA-dependent DNA polymerases. RT is a DNA polymerase enzyme that transcribes single-stranded RNA into double-stranded DNA. The enzyme is encoded and used by reverse-transcribing viruses, which use the enzyme during the process of replication. Reverse-transcribing RNA viruses, such as retroviruses (e.g., avian leukosis virus, mouse mammary tumour virus, murine leukemia virus, feline leukemia virus, bovine leukemia virus, human T-lymphotropic virus, Walleye dermal sarcoma virus, HIV-1, simian immunodeficiency virus, feline immunodeficiency virus, and chimpanzee foamy virus), use the enzyme to reverse-transcribe their RNA genomes into DNA, which is then integrated into the host genome and replicated along with it. Reverse-transcribing DNA viruses, such as the hepadnaviruses (e.g., hepatitis B), transcribe their genomes into an RNA intermediate and then, using reverse transcriptase, back into DNA.

Other features and advantages of the invention will be apparent from the Drawings, the Detailed Description, and the claims.

### BRIEF DESCRIPTION OF THE DRAWINGS

FIGS. 1A-D show the growth kinetics and viability of T cells treated with S. fusiforme. 1 G5 T cells were treated with 2 mg/ml or 4 mg/ml of S. fusiforme, with 10⁻⁶ M ddC, or were mock-treated. Total cell number (A) and the percentage of viable cells from total (B) were monitored at the indicated time points after infection by trypan blue exclusion assay by counting at least 200 cells each in three different fields under ×20 magnification using an Olympus BH-2 fluorescence microscope. The experiment was repeated with primary human peripheral blood mononuclear cells (PBMCs) treated with 1.5, 3, or 4.5 mg/ml of S. fusiforme with 10⁻⁶ M ddC, or the cells were mock-treated. Total cell number (C) and the percentage of viable cells from total (D) were monitored at the indicated time points after infection, as described above. This data shows that treatment with less than 4 mg/ml of S. fusiforme extract does not inhibit cell growth, is not toxic to cells, and is suitable for in vitro testing of HIV-1 inhibition in 1G5 T cells.

FIGS. 2A-B show the dose response of HIV-1 inhibition and cell viability in T cells treated with S. fusiforme. 1 G5 T cells were treated for 24 hours with increasing concentrations of S. fusiforme or with 10⁻⁶ M ddC, as indicated, and infected with CXCR4 tropic HIV-1 (NL4-3) at a multiplicity of infection (MOI) of 0.01 for 1.5 hours. The cells were washed 3 times and then returned to culture. On day 3 after infection, intracellular luciferase gene marker expression was measured from cell lysates adjusted to the same number of viable cells using an MTT assay (A). The percent inhibition of HIV-1 infection was calculated and plotted on the Y-axis as “% Inhibition.” In parallel, cell viability for each treatment was quantified by MTT uptake, measured at an absorbance of 570 nm (B). These results show that S. fusiforme treatment inhibits HIV-1 replication in T cells in a dose-dependent manner, inhibition is similar to that achieved with ddC treatment, and treatment is not toxic to cells.

FIGS. 3A-B show the time course of HIV-1 inhibition and viability in T cells. 1G5 T cells were treated for 24 hours with either 2 mg/ml of S. fusiforme or with 10⁻⁶ M ddC. The cells were then infected with NL4-3 at 0.01 MOI for 1.5 hours, washed 3 times, and returned to culture. On day 3 post-infection, gene expression of intracellular luciferase was measured from cell lysates adjusted to the same number of viable cells and percent inhibition was calculated and plotted on the Y-axis (A). Cell viability was determined by trypan blue exclusion assay (B). These findings demonstrate that S. fusiforme inhibits infection and de novo HIV-1 synthesis through day 7 of follow-up and this treatment does not affect cell viability.

FIGS. 4A-G show the inhibition of cell-to-cell infection and syncytia formation. Uninfected 1 G5 T cells were pretreated for 24 hours with either (A) mock-treatment, (B) 10⁻⁶ M ddC and 2 mg/ml S. fusiforme, (C) 10⁻⁶ M ddC and 4 mg/ml S. fusiforme, (D) 2 mg/ml S. fusiforme, or (E) 4 mg/ml S. fusiforme. 1 G5 cells were co-cultivated at a 1:1 ratio with CEM cells that were infected with NL4-3 at 0.01 MOI. Untreated GHOST cells were ddC treated and co-cultivated with HIV-infected 1 G5 cells and examined for syncytia formation by green fluorescence (G). Cell cultures were monitored for luciferase expression and percent inhibition was calculated from maximal luciferase expression from untreated 1 G5 cells. These results demonstrate that S. fusiforme blocks HIV-1 infection by a cell-to-cell fusion mechanism, which also prevents subsequent multinucleated cell formation and associated cytopathic effects.

FIGS. 5A-B show the inhibition of HIV-1 expression in human macrophages and microglia. Either macrophages (A) or microglia (B) were treated for 24 hours with 1 mg/ml S. fusiforme or with 10⁻⁶ M ddC, infected with primary CCR5-tropic isolate ADA at 0.2 pg of p24/cell for 2 hours, washed 3 times, and returned to culture. At the indicated time points after infection, HIV-1 expression was monitored by p24 production in cell-free supernatants by ELISA and the percent inhibition was calculated and plotted on the Y-axis. These results demonstrate that S. fusiforme is a potent inhibitor of R5-tropic HIV-1 infection in primary human macrophages and microglia and that inhibition is long lasting but not toxic to cells.

FIGS. 6A-B show the inhibition of HIV-1 infection. 1 G5 T cells were treated for 24 hours with increasing concentrations of SP4-2, with 10⁻⁶M ddC, or mock-treated, as indicated. The cells were then infected with HIV-1 (NL4-3) at a MOI of 0.01 for 1.5 hours, washed 3 times, and returned to culture. On day 3 after infection, luciferase gene marker expression was quantified from cell lysates adjusted to the same number of viable cells using an MTT assay and plotted on the Y-axis (A). The percent inhibition of HIV-1 was calculated from raw data in A and plotted on the Y-axis as “% HIV-1 Inhibition” (B). These results show that S. fusiforme is a potent inhibitor of R5-tropic HIV-1 infection in primary human macrophages and microglia. These results demonstrate that treatment with 8 pg/ml of SP4-2 is not toxic and does not affect cell growth or viability.

FIGS. 7A-C show cell growth kinetics, viability, and toxicity of SP4-2 treatment. Uninfected (A) or HIV-1 infected (B) 1G5 T cells were treated with either 8 or 24 μg/ml of SP4-2, 10⁻⁶M ddC, or were mock-treated, as indicated.

Total cell number was monitored by trypan blue exclusion assay at the indicated time points after infection by counting at least 200 cells each in three different fields under ×20 magnification using an Olympus BH-2 fluorescence microscope. In parallel, cell toxicity in both uninfected and infected cell cultures was measured by LDH release assay (C). These observations demonstrate that both ddC and SP4-2 significantly inhibit HIV-1 infection and decrease overall cell toxicity that is normally associated with active virus replication. These findings further show that S. fusiforme inhibits the virus by inhibiting the cell-to-cell spread of HIV-1.

FIG. 8 shows the inactivation of HIV-1 upon treatment with SP4-2. 32,000 infectious X4 and R5-tropic HIV-1 particles were incubated for 1 hour at 37° C. with increasing concentrations of SP4-2 or the virus was mock-treated, as indicated. Treatment was removed by centrifugation at 135,000×g for 90 minutes. Virus was resuspended in media and used to infect GHOST X4/R5 expressing cells for 4 hours at 0.3 MOI. Cultures were washed and, after 48 hours, cellular GFP expression was determined on a FACSCalibur and the data was analyzed using Cell Quest software. Percent inhibition was calculated from cells infected with mock-treated virus and plotted on the Y-axis. These results demonstrate that the S. fusiforme preparation is capable of inactivating both X4 and R5-tropic virus.

FIGS. 9A-B show the inhibition of X4- and R5-tropic HIV-1 upon administration of SP4-2. GHOST X4/R5 and GFP-expressing cells were plated at 1×10⁵/well in 12-well plates and incubated at 37° C. in a CO₂ atmosphere with increasing concentrations of SP4-2, as indicated, then infected with either X4-tropic NL4-3 (FIG. 9A, a-d) or with R5-tropic 81A (FIG. 9B, e-h) at 0.3 MOI in replicates (n=4). Two days after infection, the cells were quantified by FACS. The percentage of infected cells is shown on each panel. An uninfected and untreated control (mock) is superimposed over each graph in dotted line (representative of 4 experiments). These results show that SP4-2 inhibited both X4- and R5-tropic HIV-1 infections in a dose-dependent manner.

FIGS. 10A-H show the inhibition of HIV-1 fusion upon administration of SP4-2. SupT 1 cells (1×10⁶) were (A) mock infected, (B) infected for 2 hours at 0.5 MOI with BlaM-Vpr-X4-tropic NL4-3, (C) infected in the presence of 10 μg/ml SP4-2, or (D) infected in the presence of 250 nM AMD3100. In a parallel experiment, SupT1 cells (1×10⁶) were either (E) mock infected, (F) infected for 2 hours at 0.5 MOI with BlaM-Vpr-X4-tropic NL4-3, (G) infected in the presence of 20 ng/ml sCD4, or (H) infected in the presence of 20 ng/ml sCD4 together with 16 μg/ml SP4-2. Cells were loaded with CCF2/AM dye and fusion was analyzed by multiparameter flow cytometry using a violet laser for excitation of CCF and gated from 10,000 cells. Percentages in each panel are of cells displaying blue fluorescence (representative of 3 separate experiments). These results show that treatment with 10 μg SP4-2 inhibited HIV-1 fusion by an average of 53%. In addition, the results show that SP4-2 almost completely reversed sCD4 inhibition of HIV-1 fusion, presumably by binding to it.

FIGS. 11A-B show the inhibition of HIV-1 binding and replication upon administration of SP4-2. GHOST cells were plated at 1×10⁵/well in 12-well plates and incubated at 37° C. in a CO₂ atmosphere with increasing concentrations of SP4-2 for 1.5 hours prior to infection. Treatment was washed off 3 times with warm media and plates were transferred to 4° C. for 2 hours to cool. The cells were then infected at 4° C. with NL4-3 at 0.1 MOI for 2 hours. (A) Unbound virus was removed by washing with cold PBS, and viral particles remaining bound to the cells were quantified by p24 ELISA. (B) In a parallel experiment, infected plates at 4° C. were returned to 37° C. for 48 hours, and virus replication was quantified by p24 ELISA. Data are a mean±SD of 6 replicates. These results show that SP4-2 inhibited HIV-1 binding to cellular surface receptors in culture and inhibited virus replication in a dose-dependent manner.

FIGS. 12A-B show the inhibition of post entry HIV-1 replication upon administration of SP4-2. (A) SupT1 cells were infected for 1.5 hours in the absence of any treatment with HIV-1 chimera NL4-3 Env⁻Luc⁺/VSV-G pseudo-type, washed 3 times, and then treated with increasing concentrations of SP4-2 for 24 hours. Intracellular luciferase gene marker expression was quantified from cell lysates that were normalized to the same number of viable cells by the MTT assay, and percent inhibition of HIV-1 replication was calculated from a control cell culture of infected but untreated cells, and plotted on the y-axis. (B) A standard cell-free fluorescent reverse transcriptase (RT) assay was performed in the presence of 2 units recombinant HIV-1 RT/reaction with the indicated concentrations of SP4-2. Percent inhibition was calculated comparative to an assay performed in absence of treatment (100% RT activity). Data are a mean±SD of three separate experiments. These results demonstrate that the SP4-2 fraction inhibits the viral life cycle post-entry.

FIGS. 13A-H show the inhibition of X4- and R5-tropic HIV-1 infection. FIGS. 13A-B show flow cytometry analyses of GHOST X4/R5 GFP-expressing cells infected with (A) X4-tropic NL4-3 or (B) R5-tropic 81A, both infected at 0.3 MOI in replicate (n=3). The percentage of infected cells is indicated in each quadrant, and an uninfected and untreated control is superimposed over each histogram by a dotted line. PA μM treatment is indicated on top of each quadrant. FIGS. 13C-H show inhibition of (C-E) X4-tropic NL4-3 infection in human PBMCs and inhibition of (F-H) R5-tropic primary isolate ADA infection in human macrophages. FIGS. 13C-E show p24 antigen production on day 6 at the peak of infection in PBMCs with different treatments and infected with NL4-3 (C), the kinetics of percent inhibition throughout the infection (D), and the LD₅₀ on uninfected PBMCs treated with increasing PA concentrations (E). FIGS. 13F-H show p24 antigen production on day 10 at the peak of infection in macrophages with different treatments and infected with ADA (F), the kinetics of percent inhibition throughout the infection (G), and the LD₅₀ on uninfected macrophages treated with increasing PA concentrations (H). In both cell types, a dose-dependent inhibitory effect of PA was observed throughout productive HIV-1 infection and, most notably, at the peak of virus replication. Taken together, these results confirmed that PA isolated from S. fusiforme is the bioactive molecule responsible for the observed HIV-1 inhibition.

FIGS. 14A-C show the inhibition of HIV-1 fusion and CD4 interaction upon PA treatment. FIG. 14A shows flow cytometry analyses of SupT1 cells treated (as indicated on top of each quadrant) and infected with fusion competent BlaM-Vpr-X4-tropic NL4-3. The percent of infected cells is indicated inside each quadrant. Cells were loaded with CCF2/AM dye and fusion was analyzed by multiparameter flow cytometry using a violet laser for excitation of CCF, and gated from 10,000 cells. FIG. 14B shows a MAGI cell assay that was performed with increasing concentrations of PA treatment in the presence of 200 nM sCD4, and the percent inhibition calculated from the number of positive cells in the absence of PA treatment (0 μg). The percent inhibition was plotted on the y-axis. FIG. 14C shows dot-blot analyses of 100 μM of ¹⁴C-labeled PA (¹⁴C-PA) loaded on PVDF membrane, which was then washed and different concentrations of sCD4, ubiquitin, S100A12, and PBS were run through the vacuum. The dot-blot membrane was washed and exposed to film, which was then scanned and the dot-blot total pixel mean value (intensity*mm²) was quantified and plotted on the y-axis. These results show that PA binds to the CD4 receptor, thereby blocking virus fusion and infection

FIGS. 15A-E show in vitro binding experiments of sCD4 with PA. FIG. 15A is a homonuclear NMR spectrum of 100 μM PA in the NMR buffer (10 mM KPO₄ buffer, pH 7.0, 20% d₆-DMSO, and 80% D₂O). FIG. 15B shows the increase of the methylene STD-NMR signal of PA with the increase of the PA concentration in the sample of 14 μM sCD4 dissolved in the NMR buffer ((1) no PA; (2) molar ratio of sCD4 to PA is 1:0.1; (3) 1:0.6; (4) 1:0.8; (5) 1:1.2; (6) 1:2; (7) 1:3; (8) 1:5; (9) 1:7; and (10) 1:10). The STD-NMR signal of PA shows that PA directly binds to sCD4. FIG. 15C shows the fractional STD effect of the —(CH₂)₁₃ — signal at a given PA concentration. The gradual decrease of the STD effect indicates that the PA-sCD4 complex is specific. FIG. 15D shows a fluorescence titration experiment of sCD4 with increasing concentration of PA. Tryptophan fluorescence was measured using an excitation wavelength of 280 nm. An increase of PA causes a red shift of 2 nm and quenching of the tryptophan fluorescence of sCD4. FIG. 15E shows a binding isotherm of the normalized sCD4 tryptophan fluorescence with increasing concentration of PA at the emission wavelength of 350 nm. Curve fitting (OriginLab) using a single site binding isotherm approximation resulted in the best value for the K_{d} to be 1.5±0.2 μM. These results show that PA binds to sCD4 by utilizing its hydrocarbon chain located away from the negatively charged end of the fatty acid.

FIGS. 16A-C show inhibition of HIV-1 infection in a human cervix model of vaginal mucosa. Ectocervix tissue samples from premenopausal women were directly cultured in a non-polarized manner in 48-well plates in 300 μl/well DMEM/F 12 media for 10 days. FIG. 16A shows paraffin-embedded and hematoxylin- and eosin-(H&E) stained sections of the uninfected ectocervix tissue identified to be composed of (a) a stratified squamous epithelial cell layer, (b) a basal epithelial layer, and (c) submocosa, which was visualized with Olympus BX41 Altra 20 Soft Image System, 100× magnification. FIG. 16B shows replicates (n=6) of tissue that were treated for 24 hours with 0, 100, and 200 μM PA, and then infected with 2×10⁵ p24/ml cell-free HIV-1 BaL in 300 μl for 16 hours. Tissue was washed 3 times and returned to culture with each respective treatment for the duration of the experiment. At the indicated time points, HIV-1 replication was tested by p24 ELISA, and a two-tailed Student's t-test with p<0.05 was used to calculate statistical significance. FIG. 16C shows that, at day 10 after infection, tissue was collected and viability determined using a MTT assay (representative of 2 experiments). These results demonstrate that 200 μM PA treatment inhibits productive HIV-1 infection by up to 48% at the peak of virus replication on day 7, and that PA treatment is not toxic to tissue.

FIG. 17 shows fatty acid inhibition of reverse transcriptase. Inhibition of reverse transcription activity was determined using the HIV-1 reverse transcriptase (RT) assay kit (Invitrogen). The assay is based on the intercalation of a fluorescent dye, PicoGreen, into DNA:RNA heterodupexes. The assay was performed in accordance with the manufacturer's instructions. Briefly, two units of recombinant HIV-1 RT (Ambion) were added to a reaction mixture containing 2-fold serial dilutions of oleic or linoleic acid, as indicated. RT activity was quantified from fluorescence readings resulting from RT catalyzing RNA-DNA heteroduplex formation. Percent RT inhibition was calculated from RT reaction in the absence of either fatty acid, taken as 100% RT activity. These results demonstrate that linoleic and oleic acid are potent RT inhibitors.

### DETAILED DESCRIPTION OF THE INVENTION

The methods, kits, articles, and compositions of the invention feature a natural product, natural product extract, a fatty acid, or a compound of any of formulas (I)-(VII) for the treatment of a viral infection, e.g., HIV-1, HIV-2, or herpes (e.g., HSV-1 or HSV-2). The natural products used in the methods and compositions of the invention can include brown algae, specifically algae of the Sargassum fusiforme species. The Sargassum fusiforme algae or extracts thereof may be used to treat HIV and herpes infections.

Macrophages and T cells are major targets for HIV-1 infection. A global decline in T cell population leads to the eventual collapse of the immune system, development of the clinical manifestations of AIDS, and death of the host. Highly active antiretroviral therapy (HAART) has greatly extended the lifespan of HIV-infected individuals. However, the AIDS epidemic continues to expand globally and the long-term control of HIV-1 infection remains an elusive goal. Current HAART regimens include inhibitors of two key viral enzymes, reverse transcriptase and a viral protease. By using combinations of reverse transcriptase and protease inhibitors, dramatic reductions in the level of chronic HIV-1 viremia have been achieved in a majority of patients. However, both reverse transcriptase and protease inhibitors have significant clinical side effects. Initial optimism that the natural decay of virus-producing cells in the presence of HAART would lead to eradication of virus was short-lived. Long-term follow-up of HAART-treated individuals revealed very slow rates of decline of HIV-1 in some individuals, with continued low-level replication of virus in macrophages and T cells and viral persistence in several tissue compartments, such as the central nervous system (CNS), which is not readily accessible to current therapies.

Alternative therapies targeting HIV-specific proteins are currently being investigated. The main HIV-1 receptor, the CD4 receptor, and both co-receptors, CCR5 (R5) and CXCR4 (X4), play a pivotal role in the entry and fusion process associated with HIV-1 infection and may be useful targets for inhibition of the virus. One unique feature of the R5 co-receptor is that is contains palmitoyl moieties on three cysteine residues of the receptor's transmembrane region. Similarly, two cysteine residues of the HIV-1 envelope protein are palmitoylated. Failure of R5 to be palmitoylated results in a decrease in expression of the R5 receptor and a decrease in the life span of the R5 receptor. The presence of free intracellular or extracellular palmitic acid strongly inhibited cell-to-cell fusion during HIV-1 infection, suggesting that free palmitic acid may interfere with the fusion process, thereby inhibiting HIV infection. However, the exact mechanisms of these HIV inhibitions are unknown. Similar fatty acids, such as the unsaturated fatty acids linoleic acid and oleic acid, do not inhibit the fusion process. However, myristic acid, a saturated fatty acid, has moderate inhibitory activity. The length and degree of unsaturation appear to be crucial factors in relation to the observed antiviral activity of the fatty acid.

Applicants have shown that products derived from natural sources and palmitic acid (PA) inhibit HIV-1 infection during various stages of the life cycle. The brown algae of the class Phaeophyceae represent a potential source of novel therapeutic agents. In particular, the brown algae Sargassum fusiforme and extracts thereof contain saturated fatty acids (e.g., palmitic acid). As described herein, Sargassum fusiforme and extracts, palmitic acid, compounds of any of formulas (I)-(VIII), and combination therapies described herein can be used to block HIV or herpes infection, replication, and transmission.

Brown Algae and Extracts thereof

Any algae of the class Phaeophyceae, particularly algae of the genus Sargassum, may be used for the methods, kits, articles, and compositions of the invention described herein. Examples of brown algae from the genus Sargassum include, e.g., Sargassum fusiforme, Sargassum aquifolium, Sargassum crassifolium, Sargassum duplicatum, Sargassum filicinum, Sargassum filipendula, Sargassum gramminifolium, Sargassum henslowianum, Sargassum horneri, Sargassum ilicifolium, Sargassum mcclurei, Sargassum muticum, Sargassum myriocystum, Sargassum natans, Sargassum oligocystum, Sargassum patens, Sargassum polycystum, Sargassum serratifolium, Sargassum siliquosum, Sargassum wightii, and Sargassum vachelliannum. Additional algae that may be used in the invention described herein include, e.g., Colpomenia sinuosa, Ecklonia cava, Forsythia suspense, Laminaria digitata, Laminaria japonica, Macrocystis pyrifera, Padina arborescens, Petalonia fascia, Pilayella littoralis, Prunella vulgaris, Scytosiphon lomentaria, and Undaria pinnatifida.

Extracts of the algae may be prepared through any method known in the art for preparing an extract of a natural product. For example, an extract may be made using chemical, physical, or mechanical separation processes. The algae may be, e.g., ground and exposed to a solvent. In some instances, the solvent may be aqueous (e.g., water) or organic (e.g., acetone, methanol, or methylene chloride). The solvent may also be aqueous acetone (e.g., 70% acetone in water). The extract may be purified through any method known in the art. For example, the extract may be fractionated through, e.g., column chromatography or high-pressure liquid chromatography (HPLC). Analysis of the resulting fractions may be performed using, e.g., thin-layer chromatography (TLC) or NMR.

### Compounds of Formula (I)

The invention features methods, kits, articles, and compositions including a compound of formula (I), or a salt thereof.

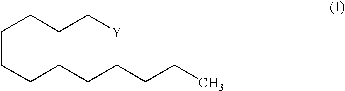

In formula (I), Y is selected from —CH₂CH₂CH₂COOH,

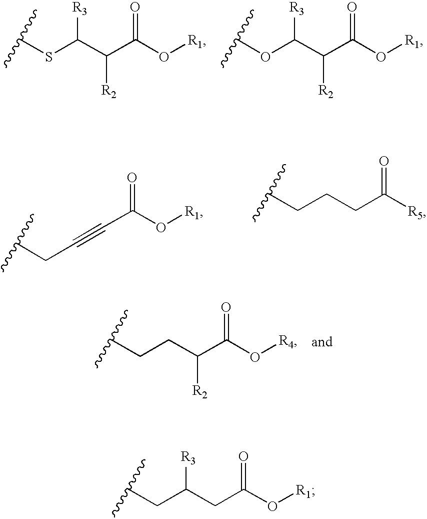

R₁ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; each of R₂ and R₃ is, independently, selected from H, —OR^{A}, —CH₃, —CH₂CH₃, halide, cyano, and nitro; R^{A} is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; R₄ is selected from C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; and R₅ is selected from H, —CH₃, —CH₂CH₃, and CF₃.

Compounds of formula (I) that can be used in the methods, kits, articles, and compositions of the invention include, without limitation, palmityl trifluoromethyl ketone (CAS 141022-99-3), 2-heptadecanone (CAS 2922-51-2; Aldrich® Cat. No. S762911), 3-octadecanone (CAS 18261-92-2; Aldrich® Cat. No. S540188), 2-hexadecynoic acid or an ester thereof, 3-dodecyloxypropionic acid or an ester thereof, 3-dodecylthiopropionic acid or an ester thereof, palmitic acid or an ester thereof, 3-hydroxyhexadecanoic acid or an ester thereof, esters of 2-hydroxyhexadecanoic acid, esters of 2-fluoropalmitic acid, and esters of 2-bromopalmitic acid.

Esters of formula (I) can be prepared, for example, from known acids using esterification techniques known in the art. Such commercially available acids include 2-hexadecynoic acid (CAS 2834-03-9), 3-dodecylthiopropionic acid (CAS 1462-52-8; Aldrich® Cat. No. S537306), palmitic acid (CAS 57-10-3; Aldrich® Cat. No. P51), 3-hydroxyhexadecanoic acid (CAS 928-17-6; Sigma® Cat. No. H4398),2-hydroxyhexadecanoic acid (CAS 764-67-0; Aldrich® Cat. No. S442240), 2-fluoropalmitic acid (CAS 16518-94-8), and 2-bromopalmitic acid(CAS 18263-25-7; Aldrich® Cat. No. 238422).

A standard facile methodology to prepare various olefinic fatty acids, which involves phosphonium salts via an autoxidation process in salt free conditions (modified Wittig reaction (see, Eynard et al., Grasas y Aceites 47:281 (1996); Poulain et al., Tetrahedron Letters 37:7703 (1996); and Sandri et al., Tetrahedron Letters 38:6611 (1997)) followed by reduction of the resulting alkene can be used to synthesize compounds of formula (I). Dodecylthiopropionic acid derivatives and dodecyloxypropionic acid derivatives can be prepared from their corresponding thiol (1-Dodecanethiol, CAS 112-55-0, Aldrich® Cat. No. 471364) and alcohol (Dodecyl alcohol, CAS 112-53-8, Aldrich® Cat. No. 126799) in a reaction to form a thioether of formula (II) and ether of formula (III), respectively. A variety of synthetic approaches are known in the art for the formation of ethers and thioethers from alcohols and thiols, respectively.

The invention features methods, kits, articles, and compositions including a compound of any of formulas (II)-(VII), or a salt thereof

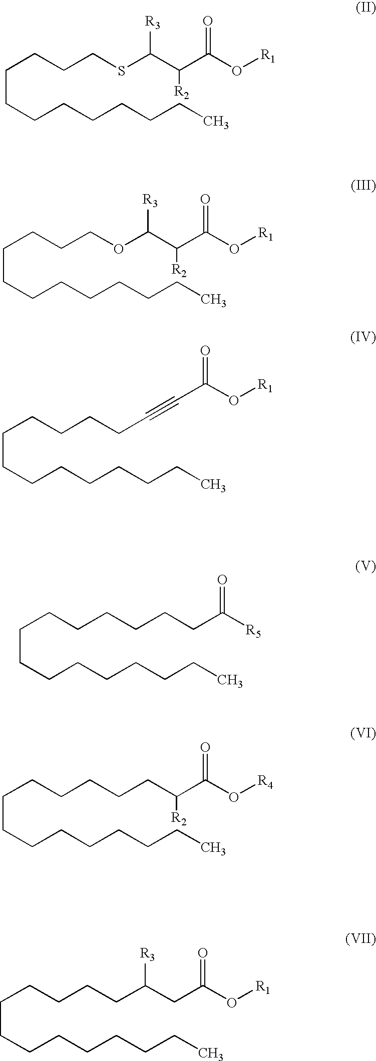

In formulas (II)-(VII), each of R₂ and R₃ is, independently, selected from H, —OR^{A}, —CH₃, —CH₂CH₃, halide, cyano, and nitro; each of R₁, R^{A}, and R₄ is, independently, selected from is selected from H, C₁₋₄ alkyl, C₂₋₄ alkenyl, C₂₋₄ alkynyl, C₂₋₅ heterocyclyl, and C₁₋₄ heteroalkyl; and R₅ is selected from H, —CH₃, —CH₂CH₃, and CF₃.

Compounds of formula (VI) which can be used in any of the methods, kits, articles, and compositions of the invention include palmitic acid, 2-fluoropalmitic acid, 2-bromopalmitic acid, 2-hydroxyhexadecanoic acid, salts thereof, and methyl or ethyl esters thereof.

### Therapy and Formulation

The pharmaceutical compositions described herein are prepared in a manner known to those skilled in the art, for example, by means of conventional dissolving, lyophilizing, mixing, granulating, or confectioning processes. Methods well-known in the art for making formulations are found, for example, in Remington: The Science and Practice of Pharmacy (21^{St} ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

Administration of compositions described herein may be by any suitable means that results in a compound concentration that is effective for treating or inhibiting HIV-1 or herpes infection. The algae, extract thereof, or fatty acid may be admixed with a suitable carrier substance, e.g., a pharmaceutically acceptable excipient that preserves the therapeutic properties of the compounds with which it is administered. One exemplary pharmaceutically acceptable excipient is physiological saline. The suitable carrier substance is generally present in an amount of 1-95% by weight of the total weight of the composition.

The compositions described herein may include, e.g., Sargassum fusiforme, an extract thereof, or a substantially pure fatty acid (e.g., palmitic acid, oleic acid, or linoleic acid). An additional therapeutic agent, e.g., an antiviral agent, may be used in therapeutically effective amounts in the methods, kits, articles, and compositions described herein.

The composition may be provided in a dosage form that is suitable for, e.g., parenteral, dermal, transdermal, sublingual, perilingual, nasal, topical administration, vaginal, rectal, and oral administration. Parenteral administration includes intravenous, intraperitoneal, subcutaneous, and intramuscular administration. Thus, the composition may be in form of, e.g., tablets, gelcaps, capsules, pills, powders, granulates, suspensions, emulsions, solutions, gels, hydrogels, oral gels, pastes, ointments, creams, plasters, drenches, delivery devices, suppositories, enemas, injectables, or implants. The composition may be sterile. The composition may be in the form of a liquid, semi-solid, or solid. The pharmaceutical composition may be, e.g., a swell-controlled, slow-release gastric, intestinal, or colonic preparation.

Pharmaceutical compositions according to the invention may be formulated to release the active compound immediately upon administration (e.g., targeted delivery), or at any predetermined time period after administration, using controlled or extended release formulations. Administration of compounds in controlled or extended release formulations is useful where the compound, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain a therapeutic level.

Many strategies can be pursued to obtain controlled or extended release in which the rate of release outweighs the rate of metabolism of the therapeutic compound. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of infection being treated, the age and condition of the patient, the stage and how the patient responds to the treatment. Additionally, a person having at risk of infection may receive treatment to inhibit transmission (i.e., to reduce their risk of infection) of a virus (e.g., HIV-1 or herpes virus).

In combination therapy, the dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds.

Each compound of the combination may be formulated in a variety of ways that are known in the art. For example, the first and second agents may be formulated together or separately. Desirably, the first and second agents are formulated together for the simultaneous or near simultaneous administration of the agents. Such co-formulated compositions can include the therapeutically active components formulated together either in a unit dosage form (e.g., in the same pill, capsule, or tablet) or non-unit dosage form (e.g., cream, liquid, or powder). By using different formulation strategies for different agents, the pharmacokinetic profiles for each agent can be suitably matched.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

### Topical Administration

Antiviral agents are desirable therapeutic modalities since, in addition to limiting viral spread in existing infection, they can also be used in microbicide formulations aimed at preventing transmission (e.g., sexual transmission) of such viruses (e.g., HIV-1 and herpes virus).

The compositions of the invention may be administered topically to an uninfected individual in an area (e.g., the penis, vagina, or rectum) that will be in contact with, e.g., a virus (e.g., HIV-1 or herpes virus) or cells infected with such a virus, prior to contact. The compositions can be topically administered in formulations that include pharmaceutically acceptable carriers, adjuvants, or vehicles, e.g., as a cream, gel, jelly, solution, or ointment, using conventional delivery systems (e.g., a suppository, sponge, diaphragm, condom, vaginal douche, or tampon). The compositions may also be topically administered to an HIV-infected individual in an area (e.g., the penis, vagina, or rectum) that will be in contact with an uninfected individual. The compositions may be topically administered prior to such contact.

The compositions and methods of the present invention may be utilized as part of a prophylactic regimen designed to inhibit or protect against viral infection (e.g., HIV-1 infection) upon, e.g., sexual contact with an infected individual. One or more compounds (e.g., palmitic acid, linoleic acid, oleic acid, any compound of any of formulas (I)-(VII) described herein, or any other compound capable of inhibiting or protecting against viral infection) can be formulated, e.g., into a cream, lotion, or douche, or may be applied into the lining of a condom. The compositions intended for vaginal application may be used in connection with presexual exposure protection. For example, the creams may be mixed with, e.g., nonoxynol-9 spermicide or added to condoms.

The prophylactically effective amount of the compositions useful in this invention that can be combined with the carrier to produce a single dosage form will vary depending upon the host being treated and the particular mode of administration. In general, the compositions of the present invention are most desirably administered at a concentration that will be sufficient to inhibit or protect against, e.g., HIV-1 or herpes infection of cells in an uninfected individual upon contact with an infected individual.

While the compositions of the invention described herein may be administered as the sole agent, the compositions may also be used in combination with one or more additional agents (e.g., antiviral agents) that are not deleterious to the activity of the compositions of the present invention or whose combination with the compounds will not have a deleterious effect on the host being treated.

In another embodiment of the method of this invention, the compositions may be added to, e.g., blood, blood by-products, a blood preparation, or other bodily fluids in vitro. The compositions can be added alone or in combination with a suitable vehicle. The effective amount required will depend upon a number of factors, including the sample and the vehicle chosen.

In further embodiments, the compositions may be added to disposable gloves, needles, or syringes used, e.g., by health care workers or researchers dealing with blood and/or bodily fluids, or to soap used in hospitals and research institutions.

### Dosages

The pharmaceutical composition described herein may be administered once, twice, three times, four times, or five times each day, or in other quantities and frequencies. Alternatively, the pharmaceutical composition may be administered once per week, twice per week, three times per week, four times per week, five times per week, or six times per week. The duration of therapy can be, e.g., one week to one month; alternatively, the pharmaceutical composition can be administered for a shorter or a longer duration. Continuous daily dosing with compounds used in the methods described herein may not be required. A therapeutic regimen may require cycles, during which time a composition is not administered, or therapy may be provided on an as-needed basis.

Appropriate dosages of compounds used in the methods described herein depend on several factors, including the administration method, the severity of the infection, and the age, weight, and health of the patient to be treated. Additionally, pharmacogenomic information (e.g., the effect of genotype on the pharmacokinetic, pharmacodynamic, or efficacy profile of a therapeutic) about a particular patient may affect the dosage used.

### Antiviral Agents

Antiviral agents which can be used in the combinations of the invention include, without limitation, abacavir, acemannan, acyclovir, adefovir, amantadine, amidinomycin, ampligen, amprenavir, atevirdine, capravirine cidofovir, delavirdine, didanosine, dideoxyadenosine, n-docosanol, edoxudine, efavirenz, emtricitabine, famciclovir, floxuridine, fomivirsen, foscarnet sodium, ganciclovir, idoxuridine, imiquimod, indinavir, inosine pranobex, interferon-α, interferon-β, kethoxal, lamivudine, lopinavir, lysozyme, madu, methisazone, moroxydine, nelfinavir, nevirapine, oseltamivir, palivizumab, penciclovir, enfuvirtide, pleconaril, podophyllotoxin, ribavirin, rimantadine, ritonavir, saquinavir, sorivudine, stallimycin, statolon, stavudine, tenofovir, tremacamra, trifluridine, tromantadine, valacyclovir, valganciclovir, vidarabine, zalcitabine, zanamivir, zidovudine, resiquimod, atazanavir, tipranavir, entecavir, fosamprenavir, merimepodib, docosanol, vx-950, and peg interferon.

One desirable antiviral agent for use in the methods, kits, articles, and compositions of the invention is acyclovir. Acyclovir is used to treat the symptoms of chickenpox, shingles, herpes virus infections of the genitals, the skin, the brain, and mucous membranes (lips and mouth), and widespread herpes virus infections in newborns. Acyclovir is also used to prevent recurrent genital herpes infections.

Structural analogs of antiviral agents which may be used in place of acyclovir in the combinations of the invention include, without limitation, 9-((2-aminoethoxy)methyl)guanine, 8-hydroxyacyclovir, 2′-O-glycyl acyclovir, ganciclovir, PD 116124, valacyclovir, omaciclovir, valganciclovir, buciclovir, penciclovir, valmaciclovir, carbovir, theophylline, xanthine, 3-methylguanine, enprofylline, cafaminol, 7-methylxanthine, L 653180, BMS 181164, valomaciclovir stearate, deriphyllin, acyclovir monophosphate, acyclovir diphosphate dimyristoylglycerol, and etofylline.

Acyclovir is currently available in cream, suspension, eye ointment, IV injection, and tablets. Acyclovir is available under the trade name Zovirax. Zovirax tablets are available in 200 mg, 400 mg, and 800 mg formulations. Zovirax cream contains 5% acyclovir. Cream excipients include, e.g., polxamer 407, cetostearyl alcohol, sodium lauryl sulphate, white soft paraffin, liquid paraffin, propylene glycol, and purified water. Combinations of the invention can be formulated in a similar fashion.

For the treatment of herpes simplex infections, Zovirax tablets (200 mg or 400 mg) are typically taken five times daily at approximately four-hour intervals, omitting the night-time dose. Treatment generally continues for 5 days, but in severe initial infections may be extended. For treatment of varicella and herpes zoster infections, Zovirax tablets (800 mg) are generally taken five times daily at approximately four-hour intervals, omitting the night-time dose, for seven days. Zovirax Cream is typically applied five times daily at approximately four-hour intervals, omitting the night-time application, for 5 days.

Penciclovir is most commonly used to treat herpes simplex viral infections, also known as cold sores. Penciclovir is available in a cream form by the trade name Vectavir or Denavir. Denavir is available for topical administration as a 1% white cream. Each gram of denavir contains 10 mg of penciclovir and the following inactive ingredients: cetomacrogol 1000 BP, cetostearyl alcohol, mineral oil, propylene glycol, purified water and white petrolatum. Denavir cream is generally applied to the affected area at approximately 2-hour intervals throughout the day for 4 days. Combinations of the invention can be formulated in a similar fashion.

Antiviral agents which can be used in the combinations of the invention include, without limitation, protease inhibitors (e.g., ritonavir, lopinavir, saquinavir, amprenavir, fosamprenavir, nelfinavir (AG1343), tipranavir, indinavir, atazanavir, brecanavir, TMC-126, darunavir, mozenavir (DMP-450), JE-2147 (AG1776), L-756423 (R-944), KNI-272, DPC-681, DPC-684, SC-52151, BMS 186318, SC-55389a, DMP-323, KNI-227, KIN-272, L697639, PL-100, PPL-100, AG-1859, RO-033-4649, GW-0385, DMP-850, DMP-851, Nar-DG-35, and BMS-232632); nucleoside reverse transcriptase inhibitors (e.g., lamivudine, zidovudine, emtricitabine, abacavir, lamivudine, zalcitabine, didanosine, stavudine, dideoxycytidine, azidothymidine, alovudine, amdoxovir, dexelvucitabine, dioxolane thymidine, elvucitabine, AVX754, DPC-817, KP-1461, MIV-210, racemic emtricitabine, GSK640385, and GSK-204937); non-nucleoside reverse transcriptase inhibitors (e.g., atevirdine, delavirdine, nevirapine, capravirine, Calanolide A, dioxolane thymidine, BILR 355BS, SJ-3366, MIV-150, GSK-695634, GSK-678248, KP-1212 and TMC-278); CCR5 inhibitors (e.g., maraviroc, aplaviroc, and vicriviroc); integrase inhibitors (e.g., raltegravir, MK-0518, GS-9137, FZ41, S-1360, L-870812, L-870810, zintevir, L731988, L708906, L731927, L731942, S-1360, L-870,812 and L-870,810);

and fusion inhibitors (e.g., enfuvirtide).

In certain embodiments, the antiviral agent used in combination with the methods, kits, articles, and compositions of the invention is selected from azidovudine (AZT), didanosine (dideoxyinosine, ddI), d4T, zalcitabine (dideoxycytosine, ddC), nevirapine, lamivudine (epivir, 3TC), saquinavir

(Invirase), ritonavir (Norvir), indinavir (Crixivan), and delavirdine (Rescriptor), among others.

### Dietary Supplements

Alternatively, a compound of formula (I), oleic acid or a salt or ester thereof, linoleic acid or a salt or ester thereof, or an extract of the invention may be administered to a subject as part of a dietary supplement, such as a vitamin supplement, mineral supplement, and/or herbal supplement.

Nutritional additives such as vitamins, vitamin components, and essential nutrients can be used for their known nutritional value as additional ingredients. Thus a vitaminic additive can include any one of, or mixtures of: vitamin A, vitamin C, vitamin D, vitamin E, vitamin K, thiamin, riboflavin, niacin, vitamin B6, folic acid, vitamin B12, biotin, and pantothenic acid, among other vitamins known in the art.

Minerals and mineral components can be used for their nutritional value as additional ingredients. Thus, a mineral additive can include any one of, or mixtures of, the following minerals or nutritionally acceptable elements thereof: calcium, copper, iron, phosphorus, iodine, magnesium, zinc, selenium, copper, manganese, chromium, molybdenum, chloride, potassium, boron, nickel, silicon tin, and vanadium, among other nutritionally important minerals known in the art.

Maintaining adequate levels of vitamins and minerals is essential to health. Many disorders due to vitamin and mineral deficiencies are well known in the art. For example, cognitive decline is a well known problem in the elderly in which diet plays a possible role. Vitamin deficiencies, especially vitamin B6, B 12 and folates, and antioxidant deficiencies (vitamins E and C) could also influence the memory capabilities and have an effect on cognitive decline (see Solfrizzi V., et al. The role of diet in cognitive decline. J. Neural Transm. 110:95 (2003)). Minerals are well known to play important roles in the maintenance of health and well-being. Selenium, for example, is a component of glutathione peroxidase, an important natural antioxidant enzyme. As another example of the importance of minerals, insufficient intake of zinc, copper, chromium, and magnesium may affect one's likelihood of developing arteriosclerosis.

Nutritional additives, such as herbs and extracts, can be used in the methods and compositions of the invention. Various processed (e.g., extracts) or unprocessed forms of the following herbs are contemplated as choices for additional nutritional ingredients in the present invention: ginseng, tea (e.g., white tea, green tea, black tea), guarana, gingko, echinacea, cinnamon, chamomile, kola nut, yerba mate, kava kava, yohimbe, elderberry, grape seed, turmeric (curcumin), milk thistle (e.g., silymarin), schisandra, panax quinquefolium, reishi, damiana, chocolate, carob, and other herbs known in the art. These herbs have been used in a variety of formulas for functional energy drinks and health drinks. Chamomile is a well-known folk remedy for insomnia and anxiety. It contains apigenin, which accounts for its anti-anxiety and sedative effects, and works in an analogous way to diazepam. Chocolate has long been known for its ability to improve mood and cognitive function. Cinnamon is known as a digestion aid that can relieve upset stomach, gas, and diarrhea. Elderberry has been shown to be active against influenza, and has long been considered a useful treatment with antiviral activity against colds, herpes, and other virus-related illnesses. Gingko biloba and its extracts have long been studied and used for the prevention and treatment of neurodegenerative pathologies. It also appears to improve mood and cognitive function in some individuals. Ginseng, in its various varieties (e.g., Asian, American, Siberian), is well known as a general health tonic that can increase physical stamina and mental alertness, counter stress, and relieve nervousness and restlessness. Grape seed extracts have been shown to have cardioprotective actions. Furthermore, animal experiments suggest that grape seed extracts can protect against ischemic neuronal damage and, thus, may have neuroprotective properties. Guarana is a common ingredient in many energy drinks and may also be used in the present invention, as can kola nuts and yerba mate. Reishi is a mushroom that has been reported to ease tension, improve memory, and sharpen concentration and focus. In an animal model, chemical constituents of schisandra have been shown to enhance cognitive function.

Any of the vitamins, minerals, herbs, and herbal extracts described herein can be used in the methods and compositions of the invention.

### Examples

### Example 1

### Inhibition of Highly Productive HIV-1 Infection in T cells, Primary Human Macrophages, and Microglia by Sargassum fusiforme

To expand our arsenal of therapeutics against HIV-1 infection, we investigated aqueous extracts from Sargassum fusiforme (S. fusiforme) to test its ability to inhibit HIV-1 infection in the periphery (e.g., in T cells and human macrophages) and in the central nervous system (CNS) (e.g., in microglia and astrocytes).

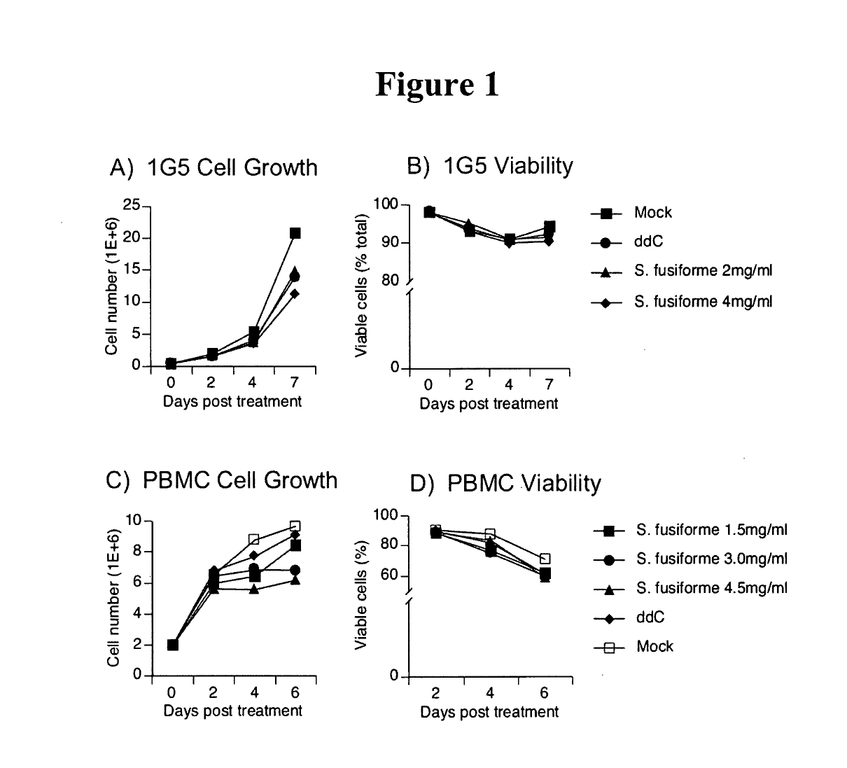

To establish a non-toxic working concentration of an S. fusiforme extract, we tested for cell growth and viability in response to treatment with S. fusiforme whole aqueous extract. T cells were treated with either 2 or 4 mg/ml of S. fusiforme extract, treated with 10⁻⁶M of the nucleoside analogue 2′,3′-didoxycytidine (ddC), or were mock-treated (FIG. 1). In 1G5 cells, the cell-growth kinetics of the extract-treated and mock-treated cells was similar. However, when cells were treated with 4 mg/ml of the S. fusiforme extract on day 7, cell growth decreased by 19% compared to mock treatment, indicating possible toxicity at this dose of the S. fusiforme extract (FIG. 1A). In parallel, we measured cell viability using trypan blue exclusion assays. In 1G5 cells and human peripheral blood mononuclear cells (PBMC), cell viability remained above 90% when treated with the S. fusiforme extract, which was comparable to mock-treated or ddC-treated cultures (FIG. 1B). We repeated this experiment with HIV-1 infected 1 G5 cells and obtained similar results. Cells treated with either 3 or 4.5 mg/ml of S. fusiforme exhibited slower growth kinetics on day 6 after treatment, as compared to the growth kinetics when cells are treated with 1.5 mg/ml of S. fusiforme extract or are mock-treated (FIG. 1C). However, viability of S. fusiforme-treated cells and mock-treated cells remained similar through day 6 after treatment. The viability of PBMCs declined over time, compared to the 1G5 T cell line (compare FIGS. 1B and 1D). Based on these results, we concluded that treatment with less than 4 mg/ml of S. fusiforme extract did not inhibit cell growth, was not toxic to cells, and was suitable for in vitro testing of HIV-1 inhibition in 1 G5 cells.

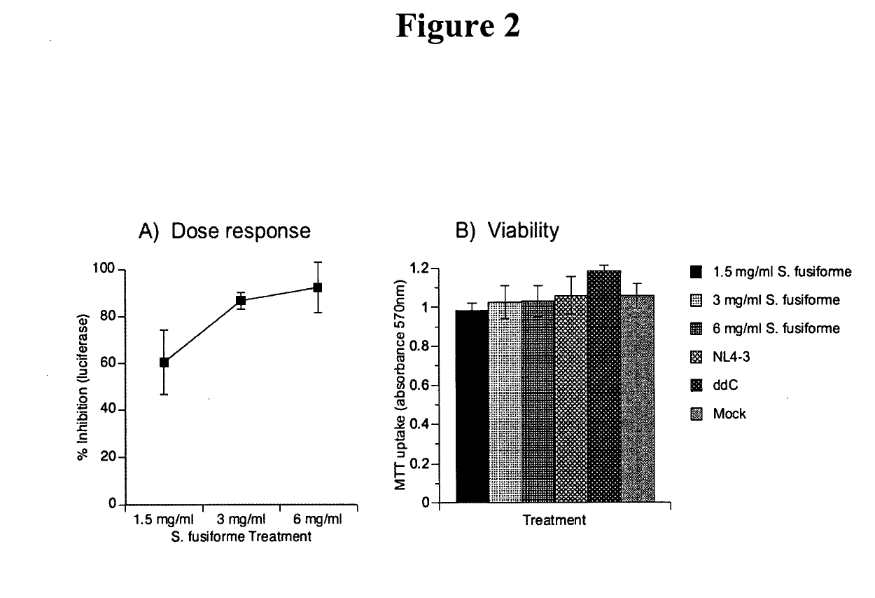

Next, we investigated the ability of S. fusiforme extracts to inhibit HIV-1 infection in 1G5 T cells. Cells were treated with increasing concentrations of S. fusiforme extract and infected with pseudotyped HIV-LTR-luciferase gene construct (NL4-3). On day 3 after infection, equal numbers of viable cells were analyzed for intracellular luciferase expression and cell viability was measured using an MTT uptake assay (FIG. 2). HIV-1 inhibition was calculated by comparing treated and untreated cell cultures (both infected with HIV-1). Treatment with 1.5, 3, and 6 mg/ml of S. fusiforme extract inhibited HIV-1 replication in a dose-dependent manner by 60.4, 86.7, and 92.3%, respectively. Treatment with ddC blocked virus replication by over 98%. In parallel, we tested for MTT uptake by viable cells, which remained high regardless of the concentration of S. fusiforme extract used in treatment (FIG. 2B). Based on these results, we concluded that treatment with S. fusiforme extract inhibited HIV-1 replication in T cells in a dose-dependent manner, inhibition was similar to that achieved with mock treatment, and treatment was not toxic to cells.

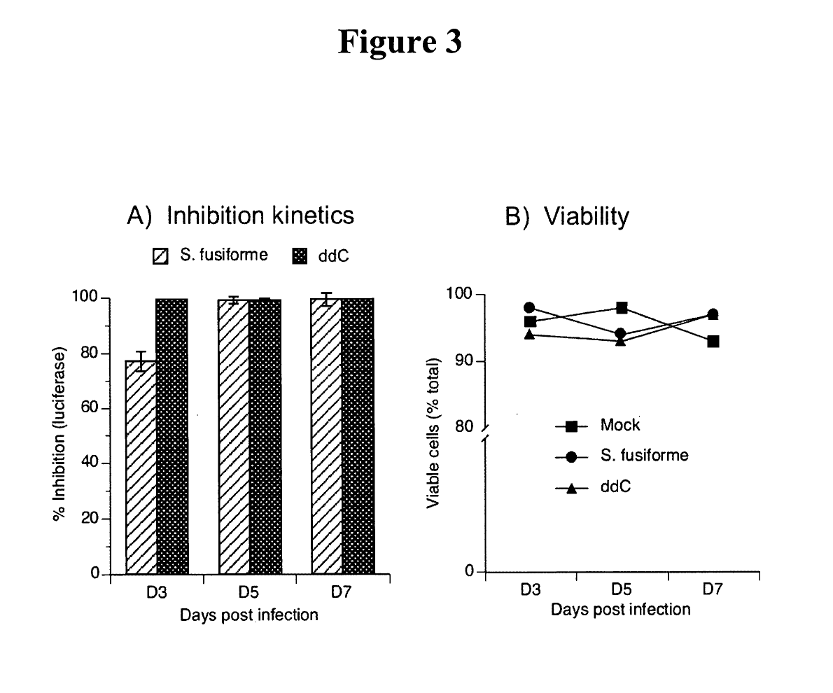

Next, we examined the duration of HIV-1 inhibition in 1 G5 T cells treated with either 2 mg/ml of S. fusiforme or with 10⁻⁶M ddC. Infection was monitored by luciferase expression at specific time points after infection (FIG. 3A). HIV-1 infection in untreated cells gradually increased from 16,110 RLU expressed on day 3 to 86,720 RLU on day 7 after infection, which demonstrated highly productive and de novo HIV-1 synthesis. Treatment with 2 mg/ml of S. fusiforme inhibited infection by 77, 99, and 99% on days 3, 5, and 7, respectively (FIG. 3A). Inhibition by ddC was 99% at each time point tested. Based on these results, we calculated the IC50 to be 0.86 mg. Similar time-course inhibition results were obtained in CEM T cells. We also tested cell viability using a trypan blue exclusion assay (FIG. 3B). Cell viability in S. fusiforme-treated cultures remained high with 98, 94, and 97% viable cells on days 3, 5, and 7, respectively. Cell viability in ddC-treated cultures was similar. Collectively, these findings demonstrated that S. fusiforme inhibited infection and de novo HIV-1 synthesis through day 7 and this treatment did not affect cell viability.

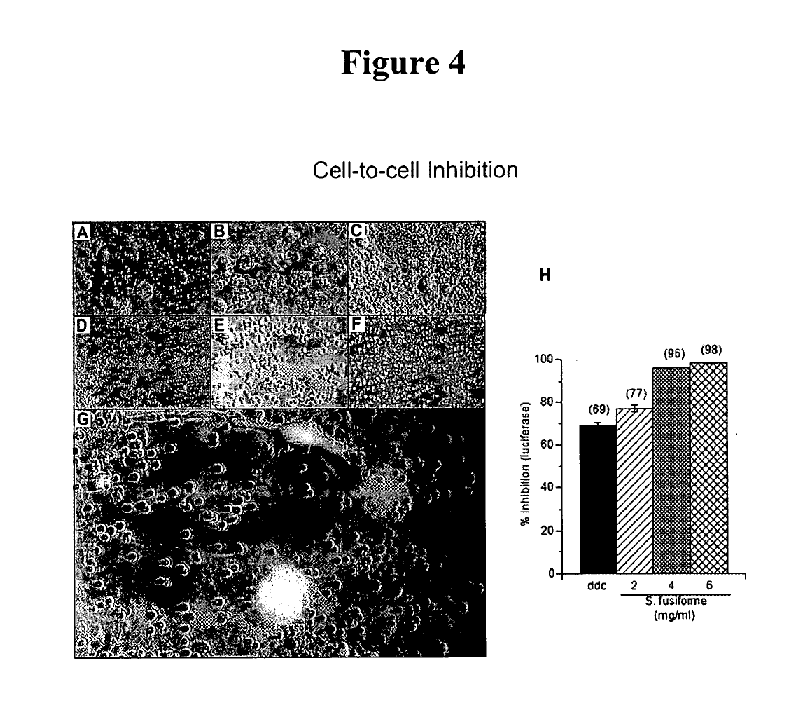

We next wanted to determine the ability of an S. fusiforme extract to block cell-to-cell mediated viral infection. To test this, we performed two separate experiments with different cell types (FIG. 4). First, we examined the ability of HIV-infected CEM cells to fuse and spread infection to uninfected 1G5 cells that were either mock-treated, treated with 10⁻⁶ M ddC only, treated with increasing concentrations of S. fusiforme extract and ddC, or treated with S. fusiforme extract only. Pretreatment of 1G5 cells with 10⁻⁶ M ddC inhibits virus replication, and therefore serves as a control for false-positive luciferase readings; however, it does not prevent the spread of infection by cell-to-cell fusion. CEM and 1 G5 cells were co-cultured for 24 hours at a ratio of 1:1 and examined for cell-to-cell fusion and syncytia formation by phase contrast microcopy or by luciferase expression. Many large syncytia were observed in co-cultures with mock-treated or ddC-treated 1G5 cells. However, treatment of 1G5 cells with 2 mg of S. fusiforme extract, with or without ddC, greatly reduced cell-to-cell fusion and syncytia formation. Cell-to-cell fusion was also inhibited in S. fusiforme-extract treated 1 G5 cells that were co-cultured with HIV-infected CEM cells. CEM cells do not have the HIV-LTR-luciferase gene and, therefore, luciferase readings from co-cultivated cell cultures can only arise from 1 G5 cells that fused with infected CEM cells. After co-cultivation with untreated 1G5 cells, luciferase expression measured 1.9×10⁵ RLU in the CEM cells, which represented maximal luciferase expression in the absence of any treatment. Treatment of 1 G5 cells with 10⁻⁶ M ddC and 2, 4, or 6 mg of S. fusiforme inhibited cell-to-cell fusion, as measured by luciferase expression in 1G5 cells, by 77, 96, and 98%, respectively. In comparison, 1G5 cell treatment with only 10⁻⁶M ddC inhibited luciferase expression by 69%. In the second experiment, we co-cultivated HIV-infected and untreated 1 G5 cells with uninfected and treated HIV-LTR-GPP-expressing GHOST adherent cells, and monitored for cell-to-cell fusion by GPP expression from GHOST cells. After co-cultivation with infected 1G5 cells, mock-treated GHOST cells can fuse and form syncytia that emit green florescence, which was detected by phase fluorescence microscopy. GHOST cells that were ddC-treated and co-cultivated with HIV-1 infected 1 G5 cells, resulted in cell-to-cell fusion and fluorescent giant cell formation. However, as in the CEM-1G5 co-cultivation experiment, no giant cells emitting green fluorescence were detected in 1G5 cells co-cultivated with GHOST cells that were treated with S. fusiforme extract. Based on the results of these experiments, we concluded that S. fusiforme extract blocks HIV-1 infection by a cell-to-cell fusion mechanism.

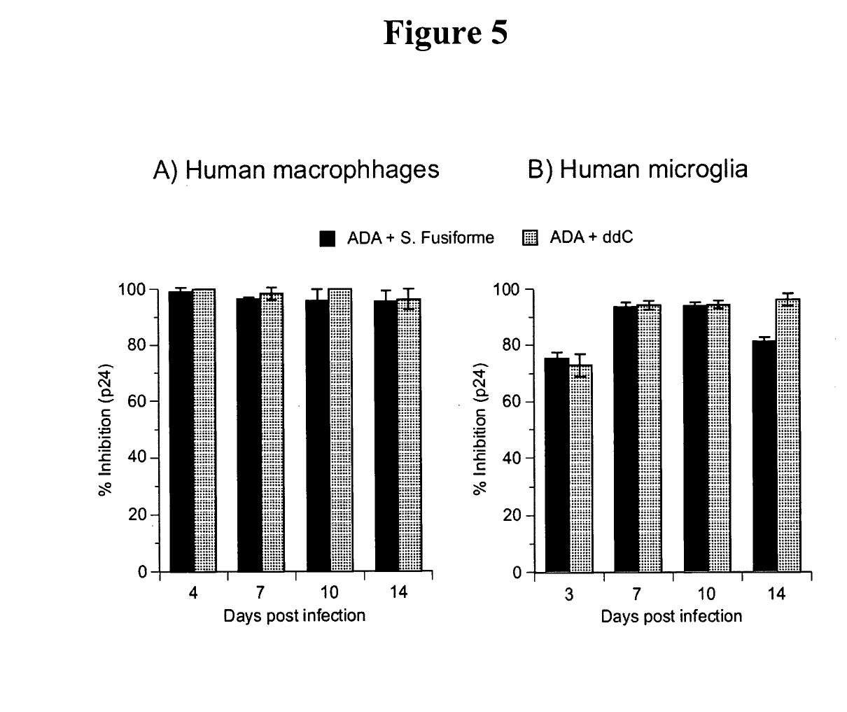

We next investigated the ability of S. fusiforme extract to inhibit virus infection in human macrophages or microglial cells. In infected and untreated macrophage cell cultures, virus levels steadily increased from day 4 to day 14, indicating productive HIV-1 infection and de novo virus synthesis. However, treatment with 1 mg/ml of S. fusiforme extract inhibited replication by over 90% through day 14 after infection, which was comparable to the inhibition with ddC treatment (FIG. 5A). Next, we treated fetal microglial cell cultures with either 1 mg/ml of S. fusiforme or 10⁻⁶ M ddC and monitored infection kinetics by p24 production in cell-free supenatants at the indicated time points after infection (FIG. 5B). As in T cells and macrophages, infected and mock-treated microglia were productively infected as demonstrated by steadily increasing p24 production that reached a peak on day 14. Treatment with S. fusiforme inhibited infection by 75% on day 3, by over 90% on day 7 and 10, and by 81% on day 14 after infection. By comparison, virus inhibition by ddC was 72% on day 3, and thereafter remained above 90%. We also monitored cell viability by MTT assay, which remained high and was similar to uninfected cell cultures. We concluded that S. fusiforme is a potent inhibitor of R5-tropic HIV-1 infection in primary human macrophages and microglia.

Collectively, our results demonstrated that S. fusiforme extract robustly inhibits HIV-1 infection in a number of cell types and in a number of infection scenarios.

### Example 2

### Biochemical Fractionation of Sargassum fusiforme Extract and its Use as a Treatment for HIV-Infected Cells

Dried S. fusiforme (1.8 kg) was ground into 40 mesh particles, soaked in 12.6 L of 70% aqueous acetone, and filtered. The extraction temperature was controlled at 70° C. to avoid possible thermal breakdown of bioactive natural products. The filtrate was concentrated to 2 L under vacuum, which was further concentrated to give a crude active extract (SP4) with an activity similar to that of the whole aqueous extract generated previously. SP4 was further fractionated by vacuum silica gel column chromatography and eluted with methylene chloride, yielding a total of 8 fractions. One of the 8 fractions (SP4-2) showed much enhanced activity in comparison with the crude extract.

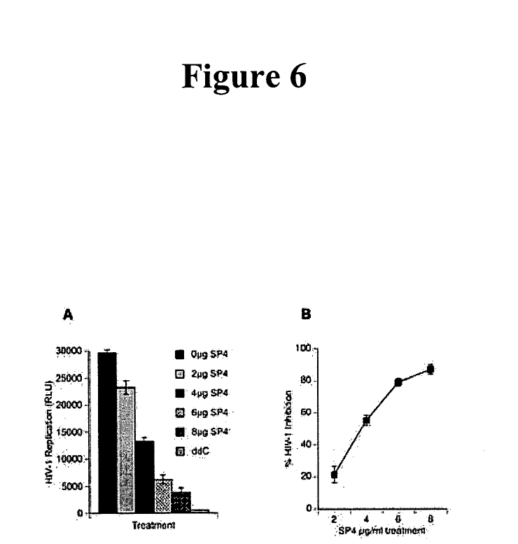

T cells were treated with increasing concentrations of SP4-2. Virus replication was measured by luciferase expression in 1G5 (FIG. 6). Maximal virus replication was determined from NL4-3 infected and mock-treated cells, which expressed 29,601 luciferase relative light units (RLU), demonstrating active and ongoing virus replication. Infection was also confirmed by flow cytometry, which revealed 99% of the cells positive for HIV-1 antigens. Comparatively, treatment with 2 μg/ml, 4 μg/ml, 6 μg/ml, and 8 μg/ml of SP4-2 reduced luciferase expression in a dose-dependent manner, to 23,243, 13,253, 6,222, and 3,877 RLU, respectively. Control cultures, treated with HIV-1 reverse transcriptase (RT) inhibitor (ddC), expressed counts of 587 RLU comparable to background values, indicating almost total inhibition of virus replication.

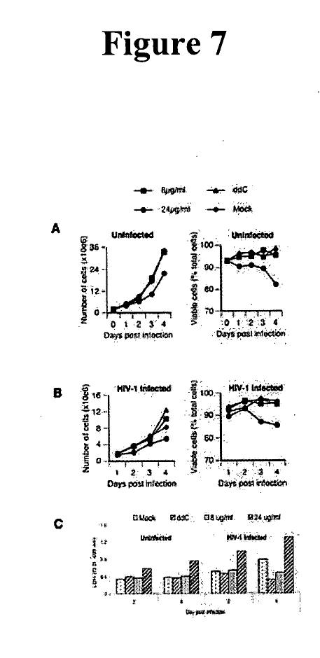

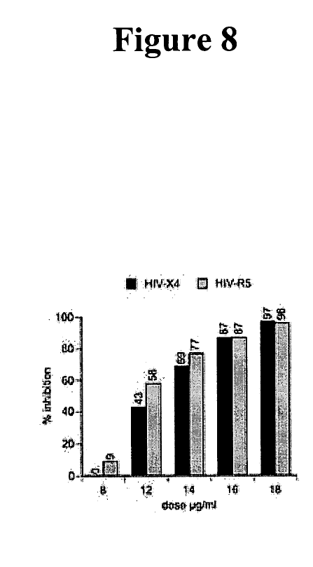

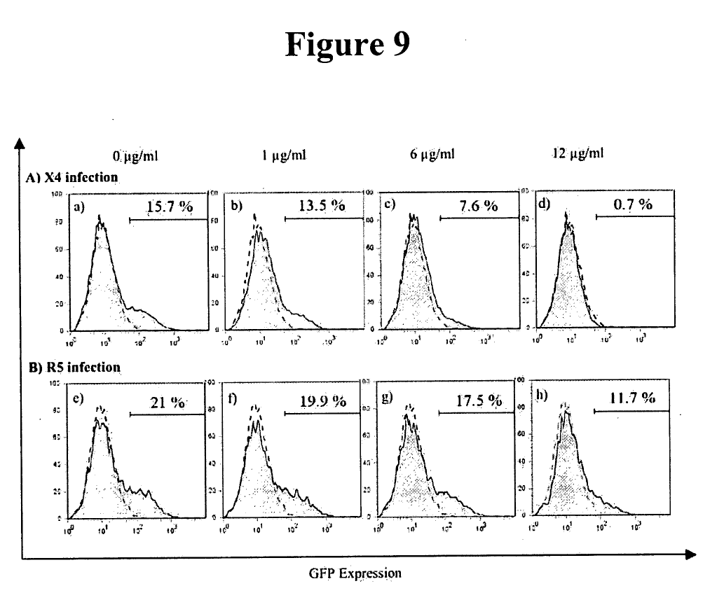

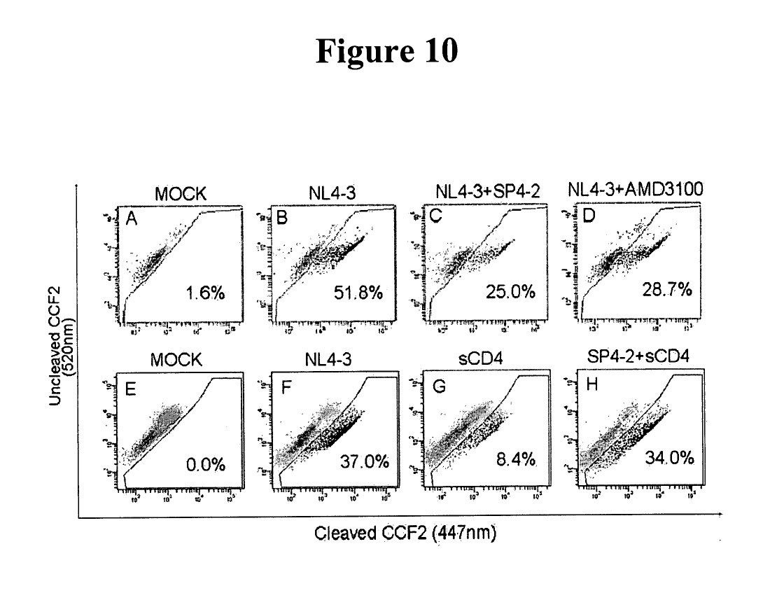

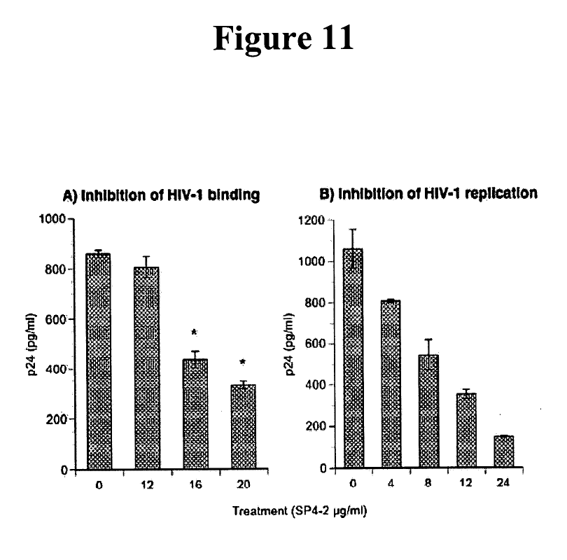

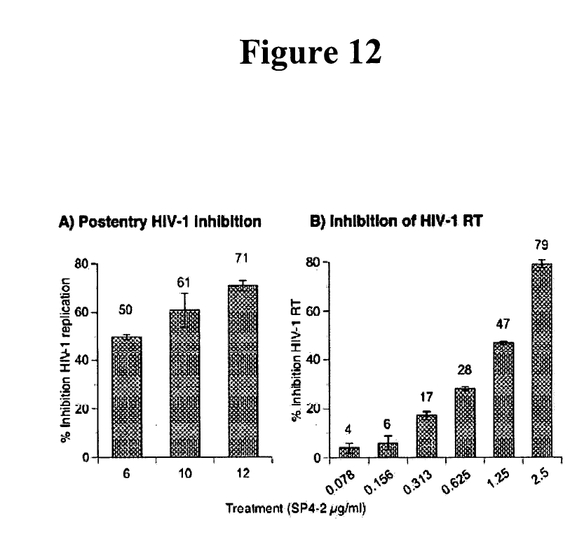

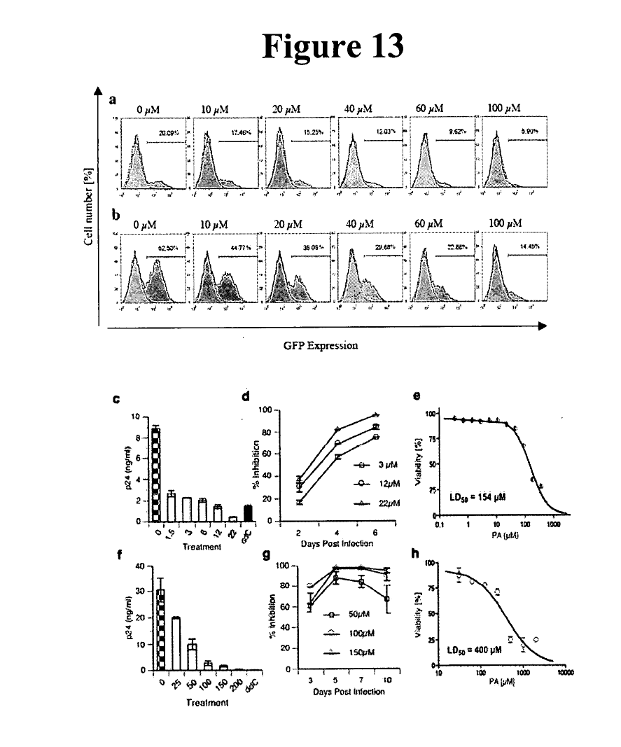

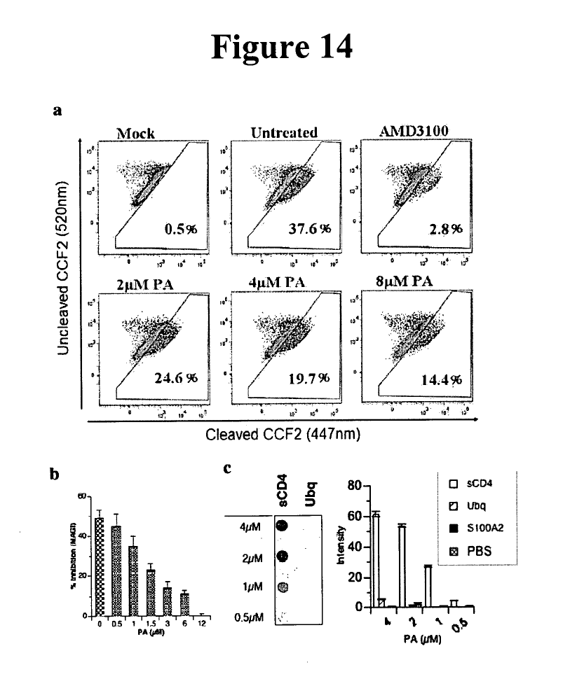

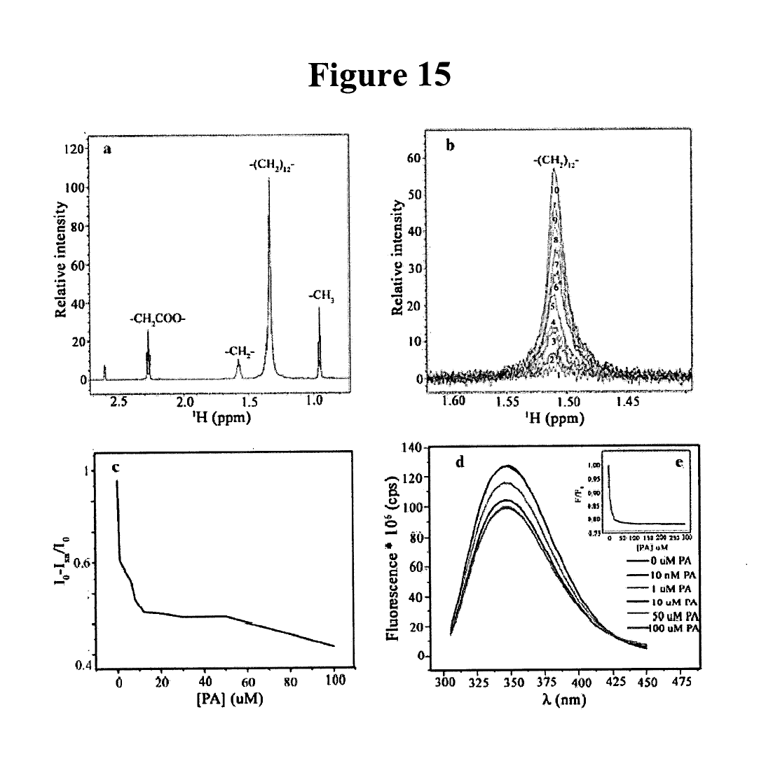

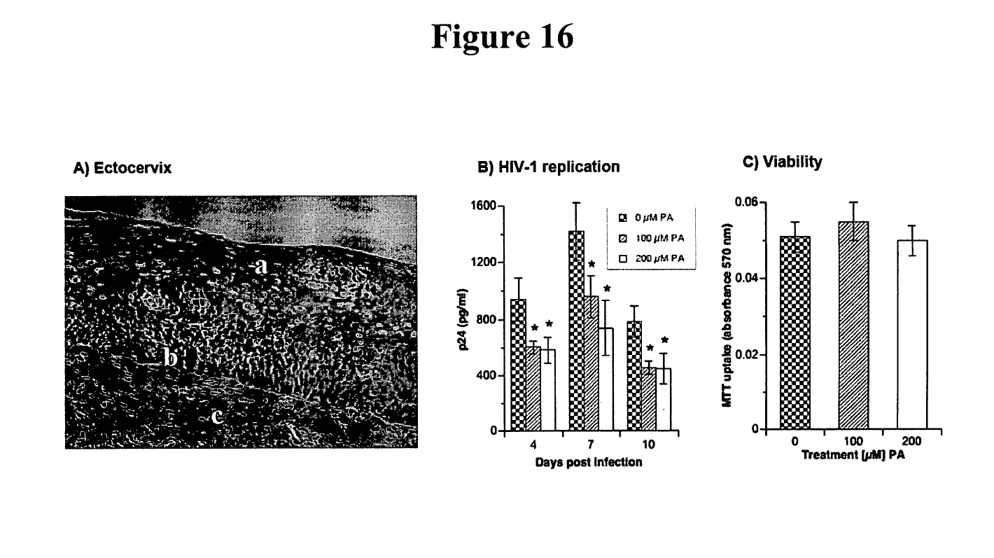

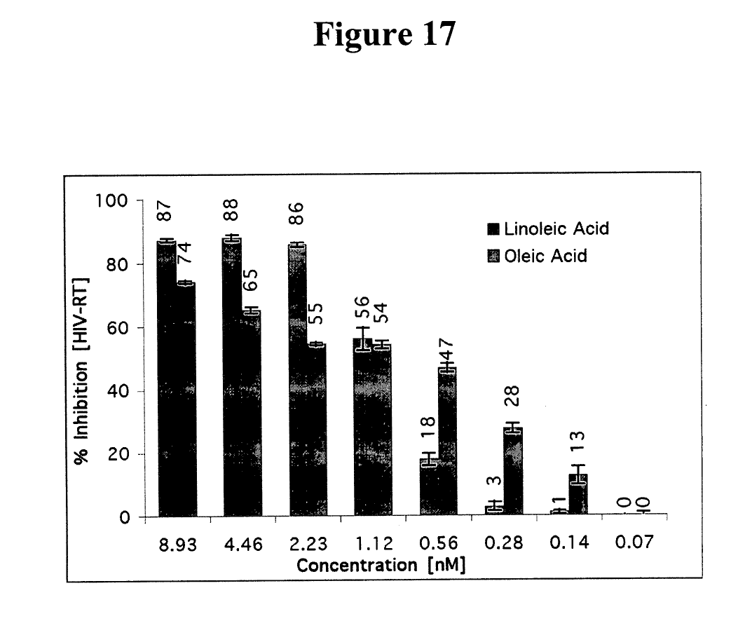

To evaluate suitability of SP4-2 for further extraction and biological activity, we treated T cells with 8 μg or 24 μg of SP4-2 or mock-treated cells, and monitored cell growth and viability by trypan blue exclusion assay and cell toxicity using quantitative LDH-release assays (FIG. 7). The experiments were performed in both HIV-infected cells and uninfected cells. Treatment with 8 μg of SP4-2, which blocked HIV-1 replication by 86%, did not inhibit cell growth or viability in either uninfected or infected cell cultures, and was similar to cultures with either ddC-treated cells or mock-treated cells (FIG. 7). In contrast, treatment with 24 μg of SP4-2 inhibited cell growth and viability in both systems. Overall slower cell growth normally associated with active virus replication was observed in infected cell cultures.

We also assayed cell toxicity by measuring LDH release from the SP4-2 treated cell cultures (FIG. 7). In the infected and uninfected cell cultures at two time points, treatment with 8 μg of SP4-2 resulted in LDH values that were similar to either mock treatment or ddC treatment, indicating no toxicity associated with these treatments. In contrast, 24 μg of SP4-2 increased toxicity in both culture systems and at both time points, which was also consistent with reduced cell growth and viability associated with the same treatment (FIG. 7). HIV-1 infection increased cell toxicity, and treatment with either 8 μg of SP4-2 or ddC, both of which inhibit virus replication, lowered this toxicity on day 4.

We next examined SP4-2 for possible virucidal activity against X4 and R5 HIV-1 (FIG. 8). We incubated 32,000 infectious HIV-1 particles with increasing concentrations of SP4-2, washed the particles by high-speed centrifugation to remove any residual SP4-2, and tested the treated virus for its ability to infect GHOST X4/R5-expressing cells. Cells were analyzed for expression of green fluorescence protein (GFP) by FACS analysis. Treatment with 8, 12, 14, 16, and 18 μg/ml of SP4-2 inhibited X4 HIV-1 infection by 0, 43, 69, 87, and 97%, and the same treatment also inhibited R5 HIV-1 infection by 9, 58, 77, 87, and 96%, respectively. These results demonstrated dose-dependent virucidal activity of SP4-2, which at 18 μg/ml inactivates both X4 and R5 HIV-1 by over 96%.

### Example 3

### Sargassum fusiforme Inhibits Both X4 and R5-Tropic HIV-1 Infection

We examined the cells' co-receptor specificity and tested the SP4-2 fraction for its ability to inhibit both X4- and R5-tropic HIV-1 (FIG. 9). GHOST cells expressing both X4 and R5 co-receptors were treated with increasing concentrations of SP4-2, and infected with X4-tropic NL4-3 (FIG. 9A) or with R5-tropic 81A (FIG. 9B), and FACS analyzed 48 hours after infection. Treatment with SP4-2 resulted in a dose-dependent decrease in the number of infected cells by either virus. X4-tropic virus (FIG. 9A) infected 15.7% cells without treatment (a), which decreased to 13.5% (b), 7.6% (c), and 0.7% (d) infected cells after treatment with 1, 6, and 12 μg/ml SP4-2, respectively. Inhibition of infection was calculated to be 14%, 51%, and 95%, respectively. For R5-tropic infection, we observed a mean of 21% infected cells (e), which decreased to 19.9% (f), 17.5% (g), and 11.7% (h) infected cells after treatment with 1, 6, and 12 μg/ml SP4-2, respectively. Inhibition of infection was calculated to be 6%, 17%, and 45%, respectively. However, when we increased SP4-2 treatment to 14, 16, 20, and 24 μg/ml, R5 inhibition of infection increased proportionally to 65%, 70%, 78%, and 88%, respectively (data not shown). Based on these results, we conclude that treatment with SP4-2 inhibits both X4 and R5-tropic HIV-1 infection in a dose-dependent manner, confirming our previous results with whole S. fusiforme extract, which inhibited both X4 and primary R5-tropic HIV-1.

### Example 4

### Sargassum fusiforme Inhibits HIV-1 Fusion by Blocking CD4 Receptor

To determine the mechanism of the observed inhibition of infection, we tested for SP4-2 activity against HIV-1 fusion to CD4-expressing SupT1 T cells by utilizing a highly specific and sensitive fluorescence resonance energy transfer (FRET)-based HIV1 fusion assay (FIG. 10). HIV-1 β-lactamase-Vpr (BlaM-Vpr) chimerical HIV-1 (NL4-3) was used to infect target cells that were loaded with CCP2/AM dye. Changes in CCP2 fluorescence reflect intracellular presence of BlaM, which is only present due to HIV-1 fusion and entry. Mock-treated negative control cells were loaded with dye, and were gated for background 520 nm emissions, which was low at 1.6% positive cells (0% fusion, FIG. 10A). After infection with BlaM-Vpr HIV-1, fusion was detected in 51.5% of the cells (100% fusion), as indicated by a shift to blue fluorescence (FIG. 10B). However, treatment of cells with 10 μg SP4-2 fraction inhibited this shift and markedly reduced viral entry with only 25% of the cells being positive for viral fusion, which corresponded to 51.7% inhibition of the fusion (FIG. 10C). As a positive control for inhibition, we treated cells with 250 nM AMD3100 (a CXCR4 inhibitor), which inhibited virus fusion, yielding 28.7% fusion positive cells that corresponded to 44.5% inhibition (FIG. 10D). Inhibition of fusion with AMD3100 increased to 80% when we increased its concentration to 500 nM (data not shown). From three different experiments, we observed that treatment with 10 μg SP4-2 inhibited HIV-1 fusion by an average of 53% (±0.8 SEM).

Next, in a parallel experiment, we studied the possible interaction between SP4-2 and CD4 (FIGS. 10E-H). BlaM-Vpr HIV-1 fusion positive cells without any inhibitor (FIG. 10F), incubated with only sCD4, resulted in 8.4% positive cells and blocked HIV-1 fusion by 77.2% (FIG. 10G). However, incubation of sCD4 together with SP4-2 resulted in 34% HIV-1 fusion positive cells (FIG. 10H), in effect reversing inhibition of fusion observed with sCD4 treatment. This result clearly indicates that SP4-2 interacts with CD4 receptor, thereby blocking HIV-1 fusion to the target cell.

### Example 5

### Sargassum fusiforme Inhibits HIV-1 Binding but Not Entry or Replication

In addition to demonstrating inhibition of HIV-1 fusion by an interaction between SP4-2 and CD4, we were interested in defining the mechanism of this inhibition by investigating whether treatment with S. fusiforme prevents virus binding to the cell surface receptors in culture (FIG. 11). Cells that are infected at 4° C. allow only HIV-1 binding to the cell surface receptor, but not fusion or entry. Except for the 2 hours of SP4-2 pretreatment of cells performed at 37° C. to allow for SP4-2-CD4 interaction, we performed all the subsequent steps, including HIV-1 infection at 4° C. GHOST X4/R5 expressing cells were treated with increasing concentrations of SP4-2 (0-20 μg), and then washed three times with warm media to remove any unbound SP4-2. Next, cells were cooled and infected at 4° C. with NL4-3 for 2 hours, washed three times to remove any unbound virus, and bound HIV-1 was quantified from replicates (n=6) by 1 HIV-1 core antigen p24 ELISA (FIG. 11A). Treatment with 0, 12, 16, and 20 μg/ml SP4-2, resulted in a dose-dependent decrease of HIV-1 bound to cells, which measured 860, 805, 435; and 331 pg/ml p24, respectively. The percent decrease in bound virus was calculated comparative to 100% bound virus (860 pg/ml p24), which was 6.3, 49.4, and 61.5%, respectively. Treatment with both 16 and 20 μg SP4-2 led to statistically significant decrease (p <0.0001) compared to no treatment (0 μg). To test whether HIV-1 bound at 4° C. was capable of membrane fusion and replication, in a parallel experiment performed under the same conditions, we returned the infected and washed cell cultures to 37° C. for 48 hours, and quantified virus replication by monitoring HIV-1 p24 production (FIG. 11B). Cell cultures pretreated with 0, 4, 8, 12, and 24 μg/ml SP4-2 replicated HIV-1 in a dose-dependent manner that produced 1061, 807, 544, 352, and 148 p24 pg/ml, respectively. HIV-1 inhibition was calculated to be 23.9, 48.7, 66.8, and 86%.

### Example 6

### Sargassum fusiforme Inhibits HIV-1 Reverse Transcriptase

We showed that inhibition by whole S. fusiforme was mediated during several stages of the virus life cycle. To determine the mechanism of this inhibition, we examined HIV-1 replication during post entry steps of the virus replication cycle (FIG. 12). HIV-1 that is envelope-deficient and is pseudotyped with VSV-G envelope bypasses any receptor entry restrictions and allows for a single round of infection, as previously demonstrated. To bypass inhibition at entry, we infected SupT1 cells with NL4-3 Env⁻Luc⁺ virus pseudotyped with VSV-G envelope for 2 hours, and then added increasing concentrations of SP4-2. Twenty-four hours after infection, we measured luciferase production and calculated inhibition of virus replication in response to SP4-2 treatment (FIG. 12A). Treatment with 6, 10, and 12 μg SP4-2 inhibited post entry HIV-1 replication in a dose-dependent manner by 50, 61, and 71%, respectively. Viability of treated cells, as quantified by a MTT assay, remained similar to mock treatment (data not shown). These data demonstrate that HIV-1 is inhibited by SP4-2 after virus entry into cells. To examine the precise mechanism of the observed post entry inhibition, we investigated direct inhibition of recombinant HIV-1 RT in a cell-free assay. Treatment with increasing concentrations of SP4-2 (0.078, 0.156, 0.313, 0.625, 0.125, and 2.5 μg) inhibited HIV-1 RT activity in a dose-dependent manner by 4, 6, 17, 28, 47, and 79%, respectively (FIG. 12B). As a negative control for inhibition, we used a different fraction that was derived from whole S. fusiforme, which was shown to be inactive during bioactivity-guided fractionation. This fraction did not inhibit HIV-1 RT (data not shown).

### Example 7

### Isolation of Four Fatty Acids from the SP4-2 Preparation from Sargassum fusiforme

A sample of S. fusiforme (14 kg) was soaked in 70% acetone (140 L×2) overnight. The filtered extract was concentrated to remove the acetone and the residue was dried overnight. The solid residue was filtered to give 75 g of a dark blue paste (SP4). SP4 (38 g) was dissolved in 200 ml of methanol and treated with 10 g of active charcoal. After filtration, the brown solution was concentrated, yielding 14 g of brown residue, which was subjected to silica gel column chromatography and eluted with methylene chloride with an increasing amount of methanol. A total of 600 fractions (25 ml/l each) were collected and grouped into 27 fractions following TLC analyses. The SP4-2 (fraction #81-120, 903 mg) was the most active fraction in an assay monitoring the inhibition of HIV-1.

SP4-2 showed one spot on silica gel TLC (methylene chloride:methanol 9.6:0.4). Both ¹H-NMR (δ, ppm, CDCl₃) and ¹³C-NMR (δ, ppm, CDCl₃) showed that SP4-2 was a mixture of saturated and unsaturated fatty acids. Palmitic acid (PA), the major component of the SP4-2 fraction, recrystallized as white plates from 10% aqueous methanol (SP4-2-P). GC-MS analysis confirmed that SP4-2 consisted of 4 fatty acids: myristic acid, palmitic acid, linoleic acid, and oleic acid.

Anti-HIV activity of the individual fatty acids was tested at four different concentrations. The values given in Table 1 represent the percentage of inhibition of cell fusion in the presence of each fatty acid.

TABLE 1

| | 0.5 μg/ml | 1 μg/ml | 2 μg/ml | 5 μg/ml |
|---|---|---|---|---|
| Myristic acid | Not tested | 8.8% | Not tested | 26.4% |
| Linoleic acid | Not tested | 0% | Not tested | 0% |
| Oleic acid | Not tested | 0% | Not tested | 0% |
| Palmitic acid | 33.5% | 46.1% | 64.2% | 50.6% |

### Example 8

### PA Inhibits X4 and R5-Tropic HIV-1 Infection in T Cells, Human PBL, and Macrophages

To test the ability of PA treatment to block productive and ongoing X4 and R5-tropic HIV-1 infection, we tested for virus inhibition in GHOST X4/R5-expressing T cells by flow cytometry (FIG. 13).

GHOST X4/R5 cells were cultured and maintained as specified by the reagent protocol. Cells were infected with HIV-1 at the indicated MOI, washed three times, and returned to culture with the indicated concentration of each treatment for the duration of experiment and then analyzed as indicated.

HIV-1 X4-tropic molecular clone NL4-3 that expresses all known HIV-1 proteins and the R5-tropic molecular clone 81A-4 that has BaL Env sequences on the backbone of NL4-3 were used for the infections in the examples described herein. We generated X4 and R5 virus as previously described. Briefly, 2.5×10⁶ 293T cells cultured in 10-cm² plates were transfected by calcium phosphate precipitation using 20 μg of NL4-3 or 81A DNA. 293T culture supernatants were harvested 48 hours after transfection, filtered through a 0.45-μm pore-size Millipore filter, and stored at −80° C. until use.

Cell-free viral stock was quantified for HIV-1 p24 using ELISA, and was also quantified for titers of infectious virus by multinuclear activation of a β-galactosidase indicator (MAGI) assay. MAGI cells were infected with NL4-3 in the presence of 200 nM sCD4 alone or incubated together with increasing concentrations of PA. Free PA was removed by filtration through a 10 kD molecular weight cut-off Microcon centrifugal membrane (Millipore) by centrifuging at 14,000×g for 30 minutes before infection, and the number of infected cells was scored 48 hours later in the MAGI assay

Culture supernatants contained 1 to 2 μg of viral p24 protein per ml and 1×10⁶ to 2×10⁶ infectious units (IU) per ml. A multiplicity of infection (MOI) of 1 for CD4-positive T cells is equivalent to approximately 1 pg of viral p24 per cell.

HIV-1 virions containing the BlaM-Vpr chimera were produced as previously described. Briefly, 293T cells in 10-cm² plates were co-transfected with pNL4-3 proviral DNA (60 μg), pCMV-BlaM-Vpr (20 μg), and pAdVAntage vectors (10 μg) (Invitrogen). After 48 hours at 37° C., the virus-containing supernatant was centrifuged at low speed to remove cellular debris and at 72,000 g for 90 minutes at 4° C. to concentrate the virus, which was resuspended in DMEM and aliquoted for storage at −80° C. For all transfections, calcium phosphate was used to precipitate DNA, and viral stocks were normalized by p24 content measured by ELISA.

For the flow cytometry analysis of GHOST X4/R5 cell HIV-1 infection of the present example, GHOST X4/R5-expressing adherent cells were stably transfected with green fluorescent protein (GFP) under control of the HIV-1 LTR promoter. Cells were plated in 24-well plates at a concentration of 5×10⁴ cells/well in DMEM, 10% FBS, 500 mg/ml G418, 100 mg/ml hygromycin, 1 mg/ml puromycin, and 1% penicillin/streptomycin. On the following day, cells were treated with dilutions of PA for 1.5 hours. PA was then removed by washing, and cells were infected at 0.3 MOI with either the X4-tropic (NL4-3) or with the R5-tropic (81A) HIV-1 clone. Infection was carried out in a volume of 150 μl at 37° C. in a 5% CO₂ atmosphere, and cell cultures were washed and returned to media containing each respective treatment. Cells were collected 40-48 hours after infection, washed in PBS, and incubated in 200 μl 1.2% paraformaldehyde in PBS for 2-3 hours at 4° C. prior to FACS analysis. Cell counting was performed on a BD FACSCanto™ FACS system and analyzed with BD FACSDiva software. The percent of infected (GFP-expressing) cells in untreated wells was taken as 100% infection, and inhibition by PA was calculated comparative to it.

In this example, cells were treated with 0, 10, 20, 40, 60, and 100 μM PA, and infected with X4 (NL4-3) or R5 (81A) HIV-1. In the X4 virus-infected cells, PA treatment reduced the total number of infected cells from 20% (0 μM) to 17.46 (10 15.25 (20 μM), 12.03 (40 μM), 9.62 (60 μM) and 5.9% (100 μM) infected cells (FIG. 13A). This reduction in the total number of infected cells translated to 13, 24, 40, 52, and 70% inhibition of X4 infection due to PA treatment. Similarly, infection with R5 virus reduced the total number of infected cells from 52.5% (0 μM) to 44.77, 36.06, 29.88, 22.88, and 14.45% infected cells, which translated to 15, 31, 43, 56, and 73% inhibition of infection (FIG. 13B). We calculated mean inhibitory concentrations (IC₅₀) for X4 and R5 inhibition to be ˜56 and 51 μM, respectively. This result demonstrated that treatment with PA blocks both X4 and R5 tropic HIV-1 infection in T cells in vitro.

Peripheral blood lymphocytes (PBL) and monocyte-derived macrophages are the primary target for HIV-1 infection and replication in vivo. Therefore, we next tested for PA inhibition of X4-tropic NL4-3 and R5-tropic ADA HIV-1 virus infection in these physiologically relevant cells (FIGS. 13C-H). Monocytes were recovered from PBMCs by countercurrent centrifugal elutriation. Monocytes were cultured as adherent monolayers (1×10⁶ cells/well in 24-well plates), differentiated for 7 days in Dulbecco's modified Eagle's medium (DMEM) supplemented with 10% human serum and macrophage colony stimulating factor (rhM-CSF). Confluent cultures of fully differentiated macrophages were infected with HIV-1 R5-tropic ADA primary isolate, as indicated in the figure descriptions. PBLs were recovered from starting PBMCs, and were cultured in RPMI and 10% fetal bovine serum, stimulated for 24 hours with 4 μg/ml PHA, cultured in the presence of 10 U IL-2, and infected with HIV-1.

In PBLs, treatment with increasing concentrations of up to 22 μM PA inhibited efficient and ongoing virus replication by 95% at the peak of infection on day 6 (FIGS. 13C-13D). We calculated the IC₅₀ to be ≈0.9 μM on day 6 (FIG. 13C) and the mean toxicity lethal dose (LD₅₀) was calculated to be ≈154 μM (FIG. 13E). Similarly, in human macrophages, treatment with up to 100 μM PA inhibited primary isolate ADA infection by 91%, on day 10 at the peak of productive virus replication (FIG. 13F-13G). The calculated IC₅₀ was ≈37 μM (FIG. 13F) and the LD₅₀ was calculated to be 400 μM (FIG. 13H). In both cell types, the dose-dependent inhibitory effect of PA was observed throughout productive HIV-1 infection and, most notably, at the peak of virus replication. Taken together, these results confirmed that PA isolated from S. fusiforme is the bioactive molecule responsible for the observed HIV-1 inhibition.

### Example 9

### PA Inhibits HIV-I Fusion

To determine the mechanism by which PA inhibits HIV-1, we next investigated the effects of PA on virus fusion and entry (FIG. 14A). HIV-fusion assays were performed as previously described. Briefly, SupT 1 cells were first infected for 2 hours with BlaM-Vpr-X4 (NL4-3) chimera at 0.5 MOI, washed in CO₂ independent media and loaded for 1 hour at room temperature (RT) with the CCF2/AM dye as specified by the manufacturer (Gibco), washed in developing buffer and the reaction was allowed to develop overnight. After development, cells were washed in PBS and fixed in 1.2% paraformaldehyde solution. BlaM reaction was detected by the change in emission fluorescence of CCF2 after cleavage by the BlaM-Vpr chimera, which was monitored by FACS with a three-laser Vantage SE (Becton Dickinson). A coherent krypton laser operating at 200 mW and generating light at 406.7 nm was used to excite the CCF2 dye. Blue emission was detected with an HQ455/50 filter, and green emission was detected with an HQ5451/90 BP filter. For light splitting, a 505 SP filter was used. Data were collected with CellQuest and analyzed with FlowJo software (Treestar).

In contrast to untreated T cells that allowed 37.6% of HIV-1 particles to enter into cells, treatment with 2, 4, and 8 μM PA restricted viral entry to 24.6, 19.7, and 14.4% virus-positive cells, respectively, which translated to 34.6, 47.8, and 61.6% inhibition of HIV-1 fusion. Inhibition was also confirmed by addition of the X4 co-receptor inhibitor AMD3100, which restricted viral entry to 2.8% of virus-positive cells.

CD4 is the main receptor responsible for HIV-1 attachment and fusion, and incubation of virus together with soluble CD4 (sCD4) protein inhibits cell infection by virtue of sCD4 competitive binding, which blocks free virus particles from attaching to the cell surface CD4 receptor. Because we previously showed that S. fusiforme extract reversed sCD4 inhibition of HIV-1 infection, presumably by binding to sCD4, we reasoned that PA might act in the same way. Indeed, incubation of sCD4 together with HIV-1 inhibited infection by 49%, and this inhibition was completely reversed by incubation of sCD4 together with increasing concentrations of PA (FIG. 14B).

Taken together, these results suggest that the PA binds to the CD4 receptor, thereby blocking virus fusion and infection. To further test this hypothesis and reveal a possible physical interaction between PA and sCD4, we performed dot blot analysis with ¹⁴C-labeled PA (¹⁴C-PA) incubated together with increasing concentrations of sCD4, ubiquitin, or S100A12 proteins (FIG. 14C). PVDF membrane (BioRad) was pretreated with 100 μM PA for 1 hour at room temperature (RT). Indicated concentrations of each protein in 10 μl/well were run through the vacuum of the dot blot apparatus. The membrane was then incubated with 100 μM ¹⁴C-labeled PA (¹⁴C-PA) in 33% ethanol for 1 hour at room temperature, and then washed in 33% ethanol and exposed to film. Blot total pixel mean value intensity was quantified by Quantity One 4.6.1 software (BioRad).

Dot blot results showed dose-dependent ¹⁴C-PA binding to increasing concentrations of sCD4. In contrast, ¹⁴C-PA did not bind to ubiquitin or to S100A12 proteins, demonstrating the specificity of the PA and sCD4 interaction, which also confirmed our results showing the ability of PA to reverse sCD4 inhibition of HIV-1 infection (FIG. 14B).

### Example 10

### PA Binds to CD4 In Vitro

We used a one-dimensional saturation transfer difference NMR (STD-NMR) experiment to characterize the binding of PA to sCD4 in vitro. STD-NMR experiments are typically used to probe for low-affinity interactions (K₄ ˜10⁻⁸ to 10⁻³M) between small molecules (e.g., ligands) and proteins, and are routinely used in drug discovery screening tests. Saturation transfer from protein-to-ligand protons identifies the binding of a ligand to a protein. Saturation transfer occurs only when the-ligand is specifically bound to the protein, and occurs at a rate that depends on the protein mobility, ligand-protein complex lifetime, and binding geometry. Protons of the ligand having the strongest contact with the protein show the most intense STD-NMR signals, enabling the mapping of the ligand's binding epitope.

To perform the STD-NMR experiment, we used commercially available sCD4 (200 μg, 4.4 nmol, Progenics Pharmaceuticals), which was exchanged into the NMR buffer (160 μl of D₂O and 40 μl d₆-DMSO, containing 10 mM KPO₄ buffer, pH 7.0). A 1 mM solution of palmitic acid was dissolved in the NMR buffer and used for the titration of the NMR sample containing 18 μM of sCD4. Approximately 20% d₆-DMSO in the NMR buffer was used to prevent formation of PA micelles. All NMR experiments were performed at room temperature. The data were acquired on a Bruker Avance 700 MHz spectrometer equipped with a z-gradient cryo probe. The on-resonance irradiation of the protein during the 1D STD NMR experiment was performed at a chemical shift of 0 ppm. Off-resonance irradiation was applied at 30 ppm. A sequence of Gauss-shaped pulses with the strength of 86 Hz and the length of 50 ms separated by a 1 ms delay was applied for 2.04 seconds during selective presaturation of the sCD4. The total number of scans was 1024. The NMR data were processed and analyzed using Topspin 2.0 (Bruker).

During the STD-NMR experiment, we titrated increasing concentrations of PA into sCD4 solution, and observed PA binding to sCD4, as shown by an increase in the intensity of the ligand STD-NMR signal (FIGS. 15A and 15B). The intensity of the STD signal was quantified by calculating the difference between the intensity of one signal in the off-resonance NMR spectrum, or reference NMR spectrum (I₀), and the intensity of a signal in the on-resonance NMR spectrum (Iₛₐₜ) for various concentrations of the ligand (FIG. 15C). As the concentration of PA increased, we observed a steady increase of the STD signal from the CH₂ and CH₃ groups of PA (FIG. 15B). Methylene groups located close to the carboxyl end of PA did not exhibit STD signal during titration. Based on these results, we concluded that PA binds to sCD4 by utilizing hydrocarbon chains located away from the negatively charged end of the fatty acid. No STD-NMR signal was observed during titration of myristic acid (MA) into the sCD4 solution (data not shown). This negative result indicates that PA binds to sCD4 specifically.

The tryptophan fluorescence of sCD4 was used to estimate binding affinity of PA to sCD4. Measurements were performed on a Fluorolog-3 fluorescence spectrophotometer (Horiba Jobin Yvon) at 25° in a 1-ml stirred cuvette. For fluorescence titration experiments, 2 μM of sCD4 (Progenics Pharmaceuticals) dissolved in the NMR buffer was used, and a 1 mM solution of PA dissolved in the NMR buffer was added in 1 μM steps. Titrations in the absence of sCD4 were performed as a reference. Tryptophan fluorescence was measured using an excitation wavelength of 290 nm. The fluorescence emission signal was subtracted from the signal of the reference titrations, and the differences adjusted by the dilution factor were plotted against the final concentration of added PA. Curve fitting (OriginLab) was performed to find the best values for the K_{d} using a single-site binding isotherm approximation.

Saturating concentrations of PA quenched 30% of the sCD4 tryptophan fluorescence and resulted in a red shift of the emission peak of 2 nm. Based on the fluorescence titration experiments (FIGS. 15D and 15E), we estimated the dissociation constant (Kd) to be 1.5±0.5 μM, which is consistent with the inhibition constants obtained from our in vivo experiments.

### Example 11

### PA Inhibits HIV-1 Infection in Human Ex Vivo Cervix Model of Vaginal Mucosa

Because of the ability of fusion inhibitors to block entry and prevent de novo infection, these therapeutic modalities are potential microbicide candidates against sexual transmission of HIV-1 infection. Considering that we demonstrated that PA is a specific CD4 fusion inhibitor, we were also interested in testing its ability to block R5-tropic HIV-1 infection in a human cervix tissue model that has been established for evaluating potential microbicides. Ectocervix tissue closely resembles the vaginal epithelial layer (FIG. 16A) and thus mimics in vivo conditions for HIV-1 infection in the female genital tract.

Ectocervix tissue samples (3-mm³ biopsy) from premenopausal women with conditions not involving cervix were obtained in accordance with AMC approved Institutional Review Board (IRB) protocol, and were processed within 1-3 hours after surgery. Tissue was cultured in a non-polarized manner in 48-well plates in 300 μl/well DMEM/F12 media (Invitrogen) supplemented with 10% FBS for the duration of the experiment. Tissue was treated with the indicated concentrations of PA and infected with HIV-1 R5 BaL at 0.3 MOI.

Before infection, cells or cervix tissue were incubated for the indicated time in culture media with different concentrations of either PA. Cells or tissue were then washed 3 times with HBSS (Invitrogen), and infected with HIV-1 at the indicated MOI. Infected cells or tissue were washed 3 times, and returned to culture with the same concentration of each treatment for the duration of experiment. Infection was monitored in cell-free supernatants by HIV-1 p24 core antigen content accumulation by enzyme-linked immunosorbent assay (ELISA) using an HIV-1 Ag kit, as specified by the manufacturer.

To test the ability of PA to inhibit HIV-1 infection in this physiologically relevant model, we treated cervix biopsy tissue with 0, 100, or 200 μM PA and then tested for inhibition of productive R5-tropic BaL infection by p24 ELISA (FIG. 16A). Measurements of HIV-1 p24 antigen levels in untreated cell-free tissue culture supernatants (0 μM) revealed a peak of p24 production on day 7 that measured 1421 pg p24/ml. This represented an increase from 943 pg p24/ml on day 4, which was followed by a gradual decline on day 10, to 785 pg p24/ml. Increasing p24 values indicated productive and ongoing HIV-1 infection and de novo viral synthesis. In contrast, treatment with 100 μM PA, significantly reduced HIV-1 replication to 604, 960, and 452 pg p24/ml on days 4, 7, and 10, respectively. Compared to untreated tissue, this reduction in HIV-1 replication was significant for each day tested (p<0.017 for day 4, p<0.049 for day 7, and p<0.014 for day 10), and it corresponded to a calculated 36, 32, and 42% inhibition of HIV-1 infection. Similar results were obtained for treatment with 200 μM PA, which was also significant when compared to untreated cultures (p<0.038, p<0.019, and p<0.029), which reduced HIV-1 infection by 38, 48, and 43% on days 4, 7, and 10, respectively (FIG. 16B). However, there was no significant difference between 100 and 200 μM PA treatment. Possible tissue toxicity was measured on day 10 after infection using a MTT viability assay, which demonstrated absence of toxicity in PA-treated tissue compared to untreated tissue (FIG. 16C). Our data demonstrate that 200 μM PA treatment inhibits productive HIV-1 infection by up to 48% at the peak of virus replication on day 7, and that PA treatment is not toxic to tissue.

### Example 12

### Inhibition of Reverse Transcriptase by Fatty Acids

Inhibition of reverse transcription activity was determined using the HIV-1 reverse transcriptase (RT) assay kit (Invitrogen). The assay is based on the intercalation of a fluorescent dye, PicoGreen, into DNA:RNA heterodupexes. The assay was performed in accordance with the manufacturer's instructions. Briefly, two units of recombinant HIV-1 RT (Ambion) were added to a reaction mixture containing 2-fold serial dilutions of oleic or linoleic acid, as indicated. RT activity was quantified from fluorescence readings resulting from RT catalyzing RNA-DNA heteroduplex formation. Percent RT inhibition was calculated from RT reaction in the absence of either fatty acid, taken as 100% RT activity (FIG. 17). These results demonstrate that linoleic and oleic acid are potent RT inhibitors.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method of treating an HIV or herpes infection in a subject in need thereof, said method comprising administering to said subject a compound of formula (I): 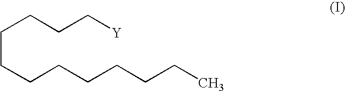
wherein Y is selected from —CH₂CH₂CH₂COOH, 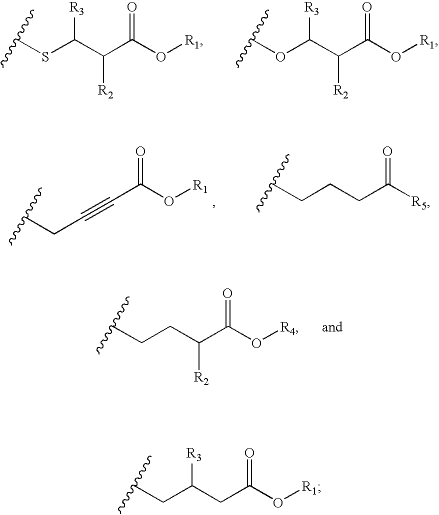
R₁ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl;
each of R₂ and R₃ is, independently, selected from H, —OR^{A}, —CH₃, —CH₂CH₃, halide, cyano, and nitro;
R^{A} is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl;
R₄ is selected from C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; and
R₅ is selected from H, —CH₃, —CH₂CH₃, and CF₃, or a salt thereof, in an amount sufficient to treat said infection.

2. The method of claim 1, further comprising administering to said subject a second compound selected from linoleic acid, salts thereof, and esters thereof, wherein said compound of formula (I) and said second compound are administered simultaneously or within 14 days of each other in amounts that together are sufficient to treat said infection.

3. The method of claim 1, further comprising administering to said subject a second compound selected from oleic acid, salts thereof, and esters thereof, wherein said compound of formula (I) and said second compound are administered simultaneously or within 14 days of each other in amounts that together are sufficient to treat said infection.

4. A method of inhibiting the transmission of HIV or herpes infection between a first subject and a second subject, said method comprising topically applying to said first subject a compound of formula (I): 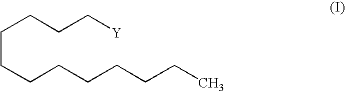
wherein Y is selected from —CH₂CH₂CH₂COOH, 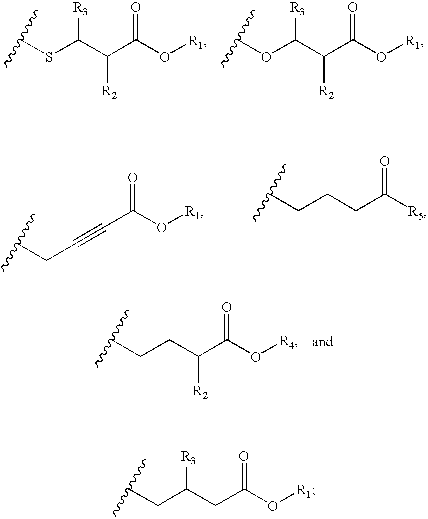
R₁ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl;
each of R₂ and R₃ is, independently, selected from H, —OR^{A}, —CH₃, —CH₂CH₃, halide, cyano, and nitro;
R^{A} is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl;
R₄ is selected from C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; and
R₅ is selected from H, —CH₃, —CH₂CH₃, and CF₃, or a salt thereof, in an amount effective to inhibit said transmission.

5. The method of claim 4, further comprising administering to said subject a second compound selected from linoleic acid, salts thereof, and esters thereof, wherein said compound of formula (I) and said second compound are administered simultaneously or within 14 days of each other in amounts that together are effective to inhibit said transmission.

6. The method of claim 4, further comprising administering to said subject a second compound selected from oleic acid, salts thereof, and esters thereof, wherein said compound of formula (I) and said second compound are administered simultaneously or within 14 days of each other in amounts that together are effective to inhibit said transmission.

7. The method of claim 1, wherein said compound of formula (I) is selected from palmityl trifluoromethyl ketone, 2-heptadecanone, 3-octadecanone, 2-hexadecynoic acid or an ester thereof, 3-dodecyloxypropionic acid or an ester thereof, 3-dodecylthiopropionic acid or an ester thereof, palmitic acid or an ester thereof, 3-hydroxyhexadecanoic acid or an ester thereof, esters of 2-hydroxyhexadecanoic acid, esters of 2-fluoropalmitic acid; and esters of 2-bromopalmitic acid.

8. The method of claim 4, wherein said compound of formula (I) is applied to the skin or a body cavity of said first subject.

9. The method of claim 8, wherein said compound of formula (I) is formulated as a foam, cream, wash, gel, spray, suppository, lotion, ointment, ovule, tampon, or aerosol.

10. The method of claim 8, wherein said compound of formula (I) is applied as part of a contraceptive device.

11. The method of claim 10, wherein said contraceptive device is an intrauterine device, intravaginal barrier, intravaginal sponge, male condom, or female condom.

12. An article comprising a compound of formula (I): 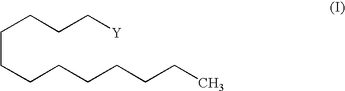
wherein Y is selected from —CH₂CH₂CH₂COOH, 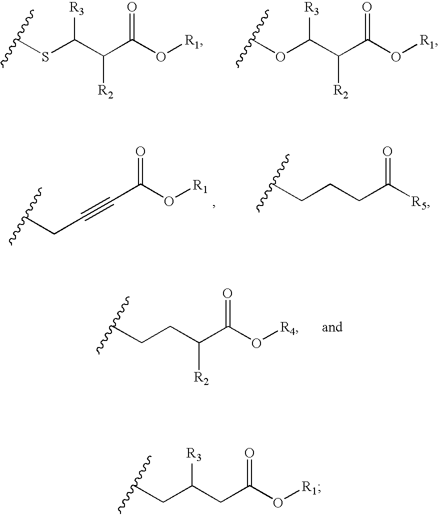
R₁ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl;
each of R₂ and R₃ is, independently, selected from H, —OR^{A}, —CH₃, —CH₂CH₃, halide, cyano, and nitro;
R^{A} is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl;
R₄ is selected from C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; and
R₅ is selected from H, —CH₃, —CH₂CH₃, and CF₃,
or a salt thereof,
in an amount sufficient to inhibit transmission of HIV or herpes to an individual wearing the article.

13. The article of claim 12, said article selected from a glove, intrauterine device, vaginal dispenser, vaginal ring, intravaginal barrier-type device, intravaginal sponge, male condom, and female condom.

14. The article of claim 12, further comprising a second compound selected from linoleic acid, salts thereof, and esters thereof, wherein said compound of formula (I) and said second compound are present in amounts that together are effective to inhibit said transmission.

15. The article of claim 12, further comprising a second compound selected from oleic acid, salts thereof, and esters thereof, wherein said compound of formula (I) and said second compound are present in amounts that together are effective to inhibit said transmission.

16. The article of claim 12, wherein said compound of formula (I) is selected from palmityl trifluoromethyl ketone, 2-heptadecanone, 3-octadecanone, 2-hexadecynoic acid or an ester thereof, 3-dodecyloxypropionic acid or an ester thereof, 3-dodecylthiopropionic acid or an ester thereof, palmitic acid or an ester thereof, 3-hydroxyhexadecanoic acid or an ester thereof, esters of 2-hydroxyhexadecanoic acid, esters of 2-fluoropalmitic acid, and esters of 2-bromopalmitic acid.

17. A pharmaceutical composition formulated for topical administration comprising from about 1% to about 50% (w/w) of a compound of formula (I): 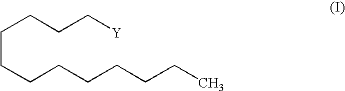
wherein Y is selected from —CH₂CH₂CH₂COOH, 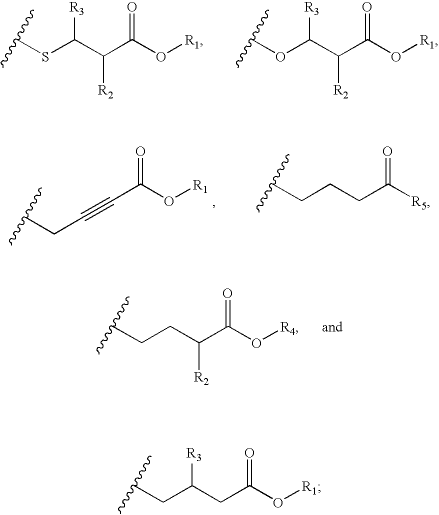
R₁ is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl;
each of R₂ and R₃ is, independently, selected from H, —OR^{A}, —CH₃, —CH₂CH₃, halide, cyano, and nitro;
R^{A} is selected from H, C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl;
R₄ is selected from C₁₋₈ alkyl, C₂₋₈ alkenyl, C₂₋₈ alkynyl, C₂₋₇ heterocyclyl, C₆₋₁₂ aryl, C₇₋₁₄ alkaryl, C₃₋₁₀ alkheterocyclyl, and C₁₋₈ heteroalkyl; and
R₅ is selected from H, —CH₃, —CH₂CH₃, and CF₃,
or a salt thereof.

18. The pharmaceutical composition of claim 17, further comprising from about 1% to about 20% (w/w) of a second compound selected from linoleic acid, salts thereof, and esters thereof

19. The pharmaceutical composition of claim 17, further comprising from about 1% to about 20% (w/w) of a second compound selected from oleic acid, salts thereof, and esters thereof.

20. The pharmaceutical composition of claim 17, wherein said compound of formula (I) is selected from palmityl trifluoromethyl ketone, 2-heptadecanone, 3-octadecanone, 2-hexadecynoic acid or an ester thereof, 3-dodecyloxypropionic acid or an ester thereof, 3-dodecylthiopropionic acid or an ester thereof, palmitic acid or an ester thereof, 3-hydroxyhexadecanoic acid or an ester thereof, esters of 2-hydroxyhexadecanoic acid, esters of 2-fluoropalmitic acid, and esters of 2-bromopalmitic acid.

21. The pharmaceutical composition of claim 17, wherein said composition is formulated as a powder, a solution, a gel, a paste, an ointment, a cream, a foam, a lotion, a plaster, a suppository, an enema, a spray, or an aerosol.

22-26. (canceled)

27. A method of treating HIV-1 infection in a subject in need thereof, said method comprising administering brown algae or an extract thereof to said subject in an amount sufficient to treat said infection.

28. (canceled)

29. The method of claim 27, wherein said brown algae are Sargassum spp.

30. The method of claim 29, wherein said Sargassum spp. is Sargassum fusiforme.

31. A method of treating HIV-1 infection in a subject in need thereof, said method comprising administering an isolated bioactive fraction of a Sargassum fusiforme extract to said subject in an amount sufficient to treat said infection.

32. The method of claim 31, wherein said isolated bioactive fraction comprises saturated fatty acids.

33. The method of claim 31, wherein said isolated bioactive fraction comprises palmitic acid.

34. The method of claim 31, wherein said extract is an aqueous extract.

35. The method of claim 31, wherein said extract is an aqueous acetone extract.

36. A method of treating HIV-1 infection in a subject in need thereof, said method comprising administering substantially pure saturated fatty acid, or a salt or ester thereof, to said subject in an amount sufficient to treat said infection.

37. The method of claim 36, wherein said saturated fatty acid is palmitic acid, or a salt or ester thereof.

38. The method of claim 37, wherein said palmitic acid, or salt or ester thereof, is isolated from an extract of Sargassum fusiforme.

39. The method of claim 1, further comprising administering to said subject an additional antiviral agent simultaneously or within 14 days of the first agent.

40. The method of claim 39, wherein said antiviral agent is a protease inhibitor, a reverse transcriptase inhibitor, an integrase inhibitor, a CCR5 antagonist, a fusion inhibitor, or a second maturation inhibitor.

41. A pharmaceutical composition comprising an isolated bioactive fraction of a Sargassum fusiforme extract and a pharmaceutically acceptable excipient.

42. The composition of claim 41, wherein said isolated bioactive fraction comprises palmitic acid, or a salt or ester thereof.

43. A pharmaceutical composition comprising substantially pure palmitic acid, or a salt or ester thereof, and a pharmaceutically acceptable excipient.

44. The composition of claim 43, wherein said palmitic acid, or salt or ester thereof, is isolated from an extract of Sargassum fusiforme.

45-53. (canceled)

54. A method of treating an HIV or herpes infection in a subject in need thereof, said method comprising administering to said subject linoleic acid, or a salt or ester thereof, in an amount sufficient to treat said infection.

55. A method of treating an HIV or herpes infection in a subject in need thereof, said method comprising administering to said subject oleic acid, or a salt or ester thereof, in an amount sufficient to treat said infection.

56. A method of treating an HIV or herpes infection in a subject in need thereof, said method comprising administering to said subject a mixture of (i) oleic acid, or a salt or ester thereof, and (ii) linoleic acid, or a salt or ester thereof, simultaneously or within 14 days of each other in amounts that together are sufficient to treat the infection.

57. A method of inhibiting the transmission of HIV or herpes infection between a first subject and a second subject, said method comprising topically applying to said first subject linoleic acid, or a salt or ester thereof, in an amount effective to inhibit said transmission.

58. A method of inhibiting the transmission of HIV or herpes infection between a first subject and a second subject, said method comprising topically applying to said first subject oleic acid, or a salt or ester thereof, in an amount effective to inhibit said transmission.

59. A method of inhibiting the transmission of HIV or herpes infection between a first subject and a second subject, said method comprising topically applying to said first subject a mixture of (i) oleic acid, or a salt or ester thereof, and (ii) linoleic acid, or a salt or ester thereof, simultaneously or within 14 days of each other in amounts that together are effective to inhibit said transmission.

60-63. (canceled)
